(19) United States
(12) Patent Application Publication
(10) Pub. No.: US 2026/0192019 A1
(43) Pub. Date: Jul. 9, 2026

(54) TISSUE-DERIVED COMPOSITIONS WITH RETAINED BIOACTIVE SUBSTANCES AND METHODS FOR MAKING AND USING SAME

(71) Applicant: Musculoskeletal Transplant Foundation, Edison, NJ (US)

(72) Inventors: Michael Zbigniew Kubik, Wallington, NJ (US); Marc Long, Monmouth Junction, NJ (US); Eric J. Semler, Morganville, NJ (US); Maitri Kapadia, Old Bridge, NJ (US)

(21) Appl. No.: 19/425,252

(22) Filed: Dec. 18, 2025

Related U.S. Application Data
(60) Provisional application No. 63/741,666, filed on Jan. 3, 2025.

(51) Int. Cl. A61L 27/36; A61L 27/54

(52) U.S. Cl. CPC A61L 27/3608; A61L 27/3695; A61L 27/54

(57) ABSTRACT

Devitalized tissue-derived compositions contain extracellular matrix (ECM), devitalized endogenous cells and/or one or more endogenous bioactive substances (e.g., cellular components, including extracellular vesicles (e.g., exosomes)) and are capable of storage at room temperature. Because native cells are devitalized, but intentionally not removed from these compositions, beneficial proteins and other substances contained in the devitalized endogenous cells and endogenous extracellular vesicles are retained in the compositions and available and accessible in quantities comparable to viable cryopreserved tissue-derived compositions and greater than conventional decellularized tissue-derived graft materials. Lyophilizing renders the devitalized tissue-derived compositions capable of storage at ambient temperatures, without the need for freezing or refrigeration. Methods for producing and using the devitalized tissue-derived compositions, either alone or with other materials and substances are also provided. In exemplary embodiments, bone tissue samples are subjected to initial freeze-thaw cycles to devitalize, but retain, endogenous cells and extracellular vesicles contained therein.

### CROSS-REFERENCE TO RELATED APPLICATIONS

The present application claims the benefit of U.S. Provisional Application No. 63/741,666, filed Jan. 3, 2025, the entire disclosure of which is incorporated by reference herein.

### FIELD OF THE INVENTION

The invention described and contemplated herein relates to tissue-derived compositions useful as grafts and which are room temperature stable. The compositions are produced from tissue samples and contain extracellular matrix and one or more bioactive substances which were endogenous to and retained from the tissue samples, and may further include undisrupted devitalized endogenous cells. The endogenous bioactive substances include, for example, bioactive factors (e.g., growth factors), and intra- or extra-cellular components (e.g., exosomes).

### BACKGROUND

The processing of tissue samples to produce tissue-derived materials suitable for use as graft material generally either intentionally removes endogenous (native) cells and their components from the tissue samples to produce decellularized tissue-derived material or maintains endogenous cells from the tissue samples in a viable state in the resulting tissue-derived material. Decellularizing typically decreases or minimizes the immunogenicity of the resulting tissue-derived materials, but often also decreases the amount of endogenous (native) bioactive substances, such as growth factors and other proteins, remaining present from the tissue samples and being available in the final tissue-derived material.

On the other hand, producing tissue-derived material which retains live, viable endogenous cells from the tissue samples sometimes increases beneficial bioactivity of the tissue-derived materials. Increased beneficial bioactivity is provided by the retained viable endogenous (native) cells and endogenous (native) materials (such as growth factors, cytokines, apoptotic bodies, and extracellular vesicles) contained in and sometimes produced by those viable endogenous cells. However, storage of such tissue-derived materials containing viable endogenous cells often requires freezing temperatures and, more particularly, deep freezing temperatures such as from −50° C. to −80° C.

Improved tissue-derived graft materials which provide beneficial bioactivity when implanted in graft recipients, but do not inconveniently require storage at temperatures below freezing, are always in demand. Processes to produce such grafts which also avoid material losses and other resource losses due to unacceptable levels of bioburden contamination would be welcomed, given the otherwise relatively high rejection rate of conventional grafts containing viable cells for such reasons since they typically need to be processed before incoming bioburden results can be obtained. The invention described and contemplated hereinafter addresses these goals by providing tissue-derived materials produced by processing one or more tissue samples and which retain one or more endogenous bioactive substances from the original tissue samples. The bioactive substances may include, but are not limited to extracellular vesicles, growth factors, and apoptotic bodies, which remain resident therein, thereon, or both, and provide increased beneficial bioactivity as compared to decellularized tissue-derived materials. Additionally, the tissue-derived materials may contain devitalized cells which are retained from and were endogenous to the original tissue samples and which contain one or more bioactive substances not yet let loose into the tissue matrix. Methods for producing such tissue-derived compositions are also provided.

### BRIEF DESCRIPTION OF THE FIGURES

The present invention will be further explained with reference to the attached drawings, wherein like structures are referred to by like numerals and/or letters throughout the several views. The drawings shown are not necessarily to scale, with emphasis instead generally being placed upon illustrating the principles of the present invention.

FIG. 1 is a chart which provides a breakdown of materials and processing conditions for several experimental test groups described in Example 1;

FIG. 2 is a graph providing the exosome content shown to be present and bioavailable in mineralized cancellous matrices produced by the experimental processing methods described in Example 3;

FIG. 3 is a graph providing data for a comparative assessment of cell devitalization, as measured by testing for presence of ATP, in various devitalized cancellous bone matrices before and after cryopreservation and lyopreservation, as described in Example 4;

FIG. 4 is an image of an H&E stained section of devitalized cancellous bone matrix for assessment of retention of cells by visualizing cell nuclei, as described in Example 4;

FIG. 5 is a graph providing data for a comparative assessment of cell devitalization in devitalized cancellous bone and fresh-cryopreserved cancellous bone, as measured by testing for presence of ATP, as described in Example 5;

FIG. 6 is a graph providing data for a comparative assessment of protein retained in fresh-cryopreserved, devitalized, and decellularized cancellous bone, respectively, as described in Example 5;

FIG. 7 is a graph providing data for a comparative assessment of cell devitalization, as measured by testing for presence of ATP, for various lyophilized devitalized cancellous bone matrices produced with apyrase treatment using varied apyrase concentrations, solution content and time, as described in Example 6;

FIG. 8 is an image of an H&E stained section of devitalized cancellous bone matrix for assessment of retention of cells by visualizing cell nuclei, as described in Example 6;

FIG. 9 is a graph providing data for a comparative assessment of cell viability in devitalized cancellous bone and fresh cancellous bone, as measured by testing for presence of ATP, as described in Example 7;

FIG. 10 is a graph providing data for a comparative assessment of the presence of angiogenic protein in mixtures of (1) devitalized cancellous bone particles and demineralized cortical bone fibers and (2) fresh cancellous bone matrix and demineralized cortical bone fibers, as described in Example 7; and

FIG. 11 is a graph providing data for a comparative assessment of the presence of exosomes, by measurement CD63, in devitalized cancellous bone and fresh viable tissue counterpart, with and without demineralized cortical bone fibers, as described in Example 7.

### DETAILED DESCRIPTION

Detailed embodiments of tissue-derived compositions and grafts comprising them are disclosed herein. It should be understood that the disclosed embodiments are merely illustrative of the invention described and contemplated herein, which may be embodied in various forms. In addition, each of any examples provided in connection with the various embodiments of the invention described and contemplated herein is intended to be illustrative, and not restrictive. Further, any figures provided are not necessarily to scale, and some features may be exaggerated to show details of particular components. In addition, any measurements, specifications and the like shown in any figures are intended to be illustrative, and not restrictive. Therefore, specific structural and functional details disclosed herein are not to be interpreted as limiting, but merely as examples for teaching one skilled in the art to variously employ the present invention. Finally, pronouns in the singular, such as “a” and “the,” are not intended to be limited to meaning that only one of the features being described is present, but rather, means that “at least one” or “one or more” such feature is present.

Generally, the invention described and contemplated herein provides tissue-derived compositions and are produced by processing one or more tissue samples. The tissue-derived compositions, as well as grafts comprising them, are useful for tissue healing, repair and reconstruction, whether for treatment of injury or disease, or for aesthetic treatments, for virtually any type of recipient including, without limitation, human, non-human mammals, marine mammals, reptiles, birds, fish, etc. More particularly, the tissue-derived compositions comprise an extracellular matrix (ECM) formed by structural matrix proteins, and either a population of devitalized endogenous cells, one or more endogenous bioactive substances, or both devitalized endogenous cells and one or more endogenous bioactive substances, resident in the ECM, all of which were endogenous to and retained from the one or more tissue samples. Bioactive substances include cellular components and bioactive factors, which will be further defined hereinbelow. Additionally, the term “devitalized tissue-derived composition” is used herein to refer to embodiments which contain a population of devitalized endogenous cells, with or without bioactive substances resident in the ECM independently of the devitalized endogenous cells.

“Retained” means that endogenous ECM, the devitalized endogenous cells, and one or more endogenous bioactive substances which were native to and present in the starting donor tissue samples, remain in the tissue during and after processing such as (without limitation) by resizing, cleaning, rinsing, demineralizing, disinfecting, sterilizing, decellularizing, dehydrating, isolating, recovering from one or more donors, etc., and are in substantially undamaged and bioactive condition in the tissue-derived compositions.

The retained endogenous bioactive substances include, but are not limited to, one or more of: cellular components, bioactive factors, and other proteins. Endogenous bioactive substances contained in the tissue-derived compositions have all been retained from the one or more tissue samples that were processed to produce the compositions. Cellular components include, but are not limited to, cytoplasm, cell membranes and other organelles such as, without limitation, nuclei which contain deoxyribonucleic acid (DNA), ribosomes which contain ribonucleic acid (RNA), vesicles which may be located inside cells or outside cells and include, without limitation, exosomes, microvesicles, and apoptotic bodies. Bioactive factors include, for example without limitation, growth factors and cytokines.

In general, vesicles may be contained within the cells of a tissue sample, resident in the ECM of a tissue sample, or both. Vesicles which are resident in the ECM (i.e., located outside cells) are referred to as “extracellular” vesicles and may be unbound or bound to the ECM. Vesicles inside cells may leave or be liberated from cells (e.g., through normal bioactivity, or as a result of apoptosis, lysing or other disruption of cell membranes), whereupon they are referred to as extracellular vesicles. Compared to vesicles bound to the ECM, unbound extracellular vesicles may be more available and readily able to promote or participate in bioactivity since they are more acutely accessible.

As already explained, the endogenous ECM, devitalized endogenous cells and/or one or more endogenous bioactive substances, are resident (present) in the tissue-derived composition and have been retained from one or more tissue samples, in substantially undamaged and bioactive condition. When describing the one or more endogenous bioactive substances, regardless of whether intracellular or extracellular, “substantially undamaged” means that at least a portion of the one or more endogenous bioactive substances which are resident and retained in the tissue-derived composition remain capable of providing the same type of beneficial bioactivity (even if not to the same degree) as they provide in fresh, unprocessed tissue samples of the same tissue type. In this context, unprocessed means the tissue sample(s) have been mechanically recovered and isolated from donor(s), may be subjected to one or more temperatures greater than zero and up to about 30° C. (i.e., not frozen, nor heated above typical room temperatures), and not chemically treated or modified such as by contact with a solution or agent other than saline or a buffered saline solution for removing debris, blood, and residual body fluids which may be present with fresh tissue samples. As applied to devitalized endogenous cells, the term “substantially undamaged” is used herein to mean that the cellular membrane is undisrupted (i.e., not lysed or otherwise open) and, therefore, contains cellular components present at the time of devitalization.

“Substantially undamaged” does not mean or require that the aforesaid endogenous components have been retained and remain in “pristine” condition in the tissue-derived compositions, i.e., they do not have to be completely undamaged or changed from their condition in the original tissue samples. Rather, “substantially undamaged and bioactive condition” describes the condition of endogenous cells, endogenous ECM, and endogenous bioactive substances, in which their natural chemical composition and physical structure are sufficiently equivalent to prior to processing so that such endogenous components are still capable of performing, participating in, promoting, enabling, facilitating, or a combination thereof, biological activity, biological mechanisms, or both.

Furthermore, the tissue-derived compositions described and contemplated herein are capable of being stored (i.e., is storable) at temperatures above freezing for a period of time, without significantly compromising its bioactivity and effectiveness. In some embodiments the tissue-derived compositions are capable of storage, for example without limitation, at room temperatures. In some embodiments the tissue-derived compositions are capable of storage, for example without limitation, at refrigeration temperatures.

The invention described and contemplated herein also relates to methods for producing the tissue-derived composition and grafts comprising them, as well as methods for their use. Generally, one or more tissue samples harvested from one or more donors (e.g., cadavers which may be human or another animal, mammalian or not), are processed under aseptic conditions to produce a tissue-derived composition.

Processing the one or more tissue samples to produce tissue-derived composition may include reducing bioburden of the one or more tissue samples to a level which makes the resulting tissue-derived composition safe for use as implantable graft material. The level or degree of bioburden reduction may be determined according to government regulation or other appropriate and accepted standards and may be accomplished using sterilizing or disinfecting techniques.

Furthermore, processing may include preserving the one or more tissue samples to produce a tissue-derived composition which is stable and storable for at least some period of time, such as at least a week and preferably even longer, with preservation of the ECM structure and bioactivity of devitalized endogenous cells, bioactive substances, or a combination thereof. Preserving may be accomplished for example, without limitation, by lyophilizing (i.e., freeze-drying), cryopreserving, contacting with a preservative (which may be in solution or not), or combinations thereof. Suitable preservatives for use in the methods described and contemplated herein for producing the tissue-derived compositions are not particularly limited and, therefore, may include any one or more of: monosaccharides (e.g., glucose, fructose, sucrose, etc.), disaccharides (e.g., trehalose), polysaccharides (e.g., dextran), anti-oxidants (e.g., vitamins, vitamin cofactors, catechins, stilbenoids, minerals), dimethyl sulfoxide (DMSO), glycerol, ethylene glycol, propylene glycol, and combinations thereof.

The processing techniques performed to produce the tissue-derived composition are designed and selected to gently remove unwanted materials (such as immunoreactive cells and substances) from the tissue samples, while retaining either devitalized endogenous cells, or one or more endogenous bioactive substances, or both, which are substantially undamaged and still bioactive, from the tissue samples. Accordingly, the tissue-derived compositions substantially lack immunoreactive cells and substances, which means the tissue-derived compositions do not cause an immune response by a recipient that would result in rejection or other complications which interfere with the desired bioactivity delivered by the tissue-derived composition after administration. As previously stated, retained endogenous bioactive substances in the tissue-derived compositions remain bioactive and include, without limitation, one or more of various cellular components, growth factors, exosomes, etc.

Furthermore, to achieve retention of endogenous ECM, devitalized endogenous cells, and/or one or more endogenous bioactive substances in substantially undamaged and bioactive condition in the tissue-derived compositions, the preservatives and any solvents used to provide solutions containing one or more preservatives which are used in the methods for making the tissue-derived compositions are biocompatible due, for example, to the properties of the solvents and preservatives themselves, the concentration(s) at which they are contacted with the tissue sample(s), the time period(s) for which they are contacted with the tissue sample(s), and combinations thereof. The resulting tissue-derived compositions should be sufficiently non-immunogenic to minimize or avoid a post-administration immune response which would result in rejection or other complications which interfere with the desired bioactivity delivered by the tissue-derived composition after administration to a recipient.

As used herein, “vesicles” means lipid bilayer-delimited particles which are naturally generated within and then subsequently released from most types of cells. Vesicles generally include exosomes, microvesicles, and apoptotic bodies. Vesicles such as exosomes contain a payload of bioactive proteins (e.g., nucleic acids (DNA), ribonucleic acid (RNA), growth factors, cytokines, etc.), lipids, metabolites, amino acids, and possibly organelles from the parent cell. Although “exosomes” are only one type of vesicle, and exosomes may be located within or outside of cells so that they may be considered extracellular vesicles or not, it is the presence and retention of endogenous vesicles from the original tissue samples that is of interest and utility here. Accordingly, to simplify discussion herein, the terms “vesicles,” “extracellular vesicles,” and exosomes, will be used herein interchangeably. Furthermore, as discussed herein, and unless otherwise specifically stated, extracellular vesicles include both those still contained in cells and those which are outside the cells and resident (whether unbound or bound) in the ECM.

Similar to known tissue-derived matrices, grafts, and compositions containing viable endogenous cells, the tissue-derived compositions described and contemplated herein are useful as grafts and, in some embodiments include a population of retained endogenous cells and their components. However, the endogenous cells retained in devitalized tissue-derived compositions are devitalized, i.e., substantially undamaged but not capable of demonstrating metabolic activity. In addition to a population of retained devitalized endogenous cells, devitalized tissue-derived compositions may also contain one or more endogenous bioactive substances (i.e., one or more bioactive factors, cellular components, or combinations thereof). Retained cellular components may, without limitation, include one or more endogenous extracellular vesicles, such as endogenous exosomes, which carry payloads of bioactive substances, as mentioned above, and are believed and known to promote or facilitate certain bioactivity that helps to achieve clinical results including without limitation, healing tissues or organs, or alleviating medical or disease conditions, particularly in high-risk patients who are compromised for normal healing.

The endogenous bioactive substances contained in and provided by extracellular vesicles may, for example without limitation, include proteins (e.g., nucleic acids, ribonucleic acid (RNA), growth factors, cytokines, etc.), lipids, one or more of which may directly and indirectly promote and facilitate the production of new tissue in-vivo. In contrast to decellularized or non-cellular tissue-derived grafts and compositions, the presence of retained, but devitalized, cells and exosomes in devitalized tissue-derived compositions may promote immunomodulation.

Additionally and without wishing to be limited by theory, it is possible that immunomodulation will be enhanced, for example without limitation, by the presence of one or more of the aforesaid bioactive substances, (whether simply retained and resident in the tissue-derived compositions, contained in devitalized cells retained and resident in the tissue-derived compositions, or both), such as by facilitating and/or promoting interaction of host immune cells, as well as bone healing/remodeling down a pro-healing/anti-inflammatory pathway. This may be particularly helpful in patients with dysregulated immune systems or chronic inflammation. Additionally, tissue-derived compositions may also promote angiogenesis or tissue genesis and provide increased levels (i.e., greater concentrations or quantities) of one or more proteins, cytokines, and growth factors.

The presence of devitalized endogenous cells and their components, including exosomes, may be confirmed and visualized in the retained endogenous ECM of tissue-derived compositions using analytical techniques involving imaging methods including, but not limited to, scanning electron microscopy, cryo-electron microscopy, and histological staining. Furthermore, immunomodulation, angiogenic, and osteogenic potential, as well as protein levels contained in the tissue-derived compositions, may be measured and compared to standard tissue-derived allografts derived from the same donors to demonstrate and confirm that the presently described tissue-derived compositions contain and provide increased levels of proteins and OIM potential.

The presence of devitalized endogenous cells in the tissue-derived compositions disclosed and contemplated herein can provide a payload of bioavailable (i.e., unbound to the ECM matrix) proteins and/or exosomes, upon disruption of such cells, while the tissue-derived compositions also retain matrix-bound exosomes and proteins that will be present for a longer duration of time. The presence and nature of matrix-bound or bioavailable (unbound) proteins and exosomes can be analyzed and compared, for example, by extraction techniques that leverage either a simple elution whereby bioavailable proteins and exosomes elute out into media or via more involved extraction processes either using chaotropic agents or enzymatic digestion to capture the matrix-bound fraction.

While the following detailed description of the tissue-derived compositions and methods for producing them is directed to bone tissue-derived compositions which retain one or more endogenous bioactive substances and possibly a population of devitalized endogenous cells and, more particularly, is directed to such compositions wherein the bone tissue samples comprise cancellous tissue samples, the presently described and contemplated invention is not limited to embodiments produced from bone tissue samples. Rather, the tissue-derived compositions and methods for making them which are described and contemplated herein are intended and expected to include embodiments produced from samples of other types of tissue and organs.

Suitable tissue types for the one or more tissue samples, include, but are not limited to, one or more of: adipose, amnion, blood vessels, bone marrow, bone, cartilage, chorion, dermis, fascia, heart valves, intervertebral disc, intestinal mucosal tissue, intestinal serosal tissue, ligament, marrow, meniscus, muscle, nerve, pericardium, perichondrium, periosteum, peritoneum, placental disc, tendon, umbilical cord, and Whartons jelly. One or more organs and ECM derived therefrom which are suitable for such processing include, without limitation, bladder, heart, kidney, liver, lung, and pancreas.

Furthermore, the methods for making tissue-derived compositions that are described and contemplated herein, may include other processing steps and techniques understood by persons of ordinary skill in the relevant art to be applicable and effective for processing tissue samples to produce the tissue-derived compositions from tissue samples which contain viable cells in their normal state and are susceptible to one or more processing treatments and steps including, but not limited to, separating tissue, combining tissue, reducing size, modifying shape, modifying immunogenicity, decellularizing, rinsing, cleaning, soaking, disinfecting, sterilizing, dehydrating, and others as are effective and beneficial for tissue processing.

Furthermore, the tissue-derived compositions described and contemplated herein are not limited to each of them having been derived from a single type of tissue or limited to including only tissue-derived materials. Rather, it is within the scope of the presently described and contemplated invention to include tissue-derived compositions which comprise and have been derived from tissue samples of more than one type of tissue, as well as one or more tissue-derived compositions having devitalized cells and/or one or more bioactive substances.

Furthermore, one or more of the tissue-derived compositions may be mixed, layered, coated, affixed, or otherwise combined with each other to provide compositions useful as grafts which are capable of providing the capabilities and benefits of the tissue-derived compositions. One or more other materials or components (e.g., other processed or unprocessed natural materials, synthetic materials, textiles, carriers, and combinations thereof, etc.) may be mixed, added, connected, affixed, adhered, adsorbed, infused, or otherwise combined with one or more of the tissue-derived compositions to provide compositions useful as grafts which are capable of providing the capabilities and benefits of the tissue-derived compositions. Processed or unprocessed natural materials include, but are not limited to, one or more tissue-derived matrices and fluids, cellulose and its derivatives, collagen, and many other materials as are determinable by persons of ordinary skill in the relevant art.

Such additional materials may be combined with the tissue-derived composition during processing to produce a graft, or before or at the time of use by a practitioner. For example, one or more carriers, including but not limited to; a sodium chloride solution, a physiological salt solution (phosphate buffered saline; PBS), blood, plasma, bone marrow aspirate (BMA), platelet-rich plasma (PRP), stromal vascular fraction (SVF)), corticosteroid, a solution containing hyaluronic acid (HA) or anti-inflammatory agents, balanced salt solution (BSS), one or more antibiotic agents (in solution or not), comparable biocompatible fluids and solutions, and combinations thereof, may be combined with the tissue-derived composition at the time of use. Other types of suitable carriers include, but are not limited to, collagen-based carriers (e.g., gelatin), hydrogels, and one or more polymers (e.g., polyethylene glycol (PEG), polylactic acid (PLA), polyglycolic acid (polyglycolide, PGA), poly(lactic-co-glycolic) acid (PLGA), reverse phase compounds and polymers medium).

In exemplary embodiments described in more detailed below, the tissue-derived compositions comprise endogenous bone tissue matrix (bone tissue ECM) derived from cancellous bone tissue samples and containing one or more endogenous bioactive substances, such as growth factors and exosomes, with or without devitalized endogenous cells, each of which has been retained in substantially undamaged and bioactive condition from the one or more tissue samples processed to produce the compositions.

Regardless of the tissue types of the one or more tissue samples processed to produce the tissue-derived composition, their physical form is not particularly limited. The physical form of the tissue-derived composition may, for example without limitation, be selected based on the anticipated or intended use or procedure, the body feature to be treated, the anticipated or intended method or technique for administration or delivery of the tissue-derived composition. Given such considerations, and notwithstanding exemplary embodiment described herein as comprising devitalized cancellous granules, particles, fibers, etc., the tissue-derived composition may have any of one or more possible physical forms, including but not limited to: granules, particles, fibers, plugs, wedges, pins, chips, strips, pieces, sheets, as well as other regular or irregular three-dimensional geometric shapes (i.e., monolithic piece, agglomerated or otherwise cohesive mass, brick, sphere, ovoid, arcuate, having contours, linear or rounded edges and corners, etc.).

Optionally, the tissue-derived compositions described and contemplated herein may further comprise additional components such as, without limitation, one or more allograft tissue-derived materials, such as non-demineralized, partially demineralized or demineralized cortical bone fibers and/or particles, and/or cancellous bone particles (e.g., granules, powder, etc.), as well as other materials produced from other types of tissue. Bone-derived compositions as described and contemplated herein are expected to possess beneficial characteristics including, but not limited to, rapid wettability, excellent handling properties, no requirement for terminal sterilization, capable of being shipped and stored at ambient temperatures.

In some embodiments, tissue-derived compositions comprising cancellous bone-derived granules or particles (or a mixture thereof) which contain one or more bioactive substances, may be combined with cortical bone fibers to produce a graft. In some embodiments, a graft may comprise one or more such bone-derived compositions, as well as cortical bone fibers, cortical bone particles (e.g., granules, powder, etc.), or a combination thereof, and they may be demineralized, partially demineralized, non-demineralized, or combinations thereof.

In some embodiments, tissue-derived compositions comprising cortical bone-derived fibers, granules, or particles (or a mixture thereof) which contain one or more bioactive substances, may be combined with cancellous bone granules or particles (or a mixture thereof) to produce a graft.

In some embodiments, a graft comprising cancellous bone-derived granules or particles (or a mixture thereof) which contain one or more bioactive substances, may also comprise demineralized cortical bone fibers and, optionally, granules or particles of non-demineralized cancellous bone, non-demineralized, cortical bone, or a combination thereof.

In some embodiments, a graft comprising cancellous bone-derived granules or particles (or a mixture thereof) which contain one or more bioactive substances, may be further comprise any one or more of: demineralized cortical bone particles, demineralized cortical bone fibers, non-demineralized cancellous bone granules or particles (or a mixture thereof), non-demineralized cortical bone particles or fibers, or a combination of both.

When implanted, such grafts are expected to provide bioactivity comparable to or the same as cancellous bone samples prior to devitalizing and other processing. Physical characteristics of the tissue-derived compositions and grafts which include them, such as but not limited to cohesiveness, moldability and reshapability, flowability, injectability, shape retention after (re)hydration, irrigation resistance, migration resistance, and degradation rate, may be determined, modified, or both, by selection of the additional tissue-derived and other materials combined with the tissue-derived compositions.

For example, without limitation, in some embodiments, devitalized tissue-derived compositions comprising devitalized cancellous bone-derived granules or particles, may be combined with demineralized cortical bone fibers to produce a graft. In some embodiments, graft materials may comprise one or more devitalized tissue-derived compositions, as well as cortical bone fibers, cortical bone particles (e.g., granules, powder, etc.), or a combination thereof, and they may be demineralized, partially demineralized, non-demineralized, or combinations thereof. Physical characteristics, such as but not limited to cohesiveness, moldability and reshapeability, flowability, injectability, shape retention after (re)hydration, irrigation resistance, migration resistance, degradation rate, may be determined or modified, or both, in such embodiments via selection of the additional tissue-derived and other materials combined with the tissue-derived compositions.

In some embodiments, devitalized tissue-derived compositions comprising devitalized cancellous bone granules or particles, may be combined with demineralized cortical bone fibers and, optionally, granules or particles of non-demineralized cancellous bone, non-demineralized, cortical bone, or a combination of both, to produce graft material.

In some embodiments, devitalized tissue-derived compositions comprising devitalized cancellous bone granules or particles, may be combined with any one or more of demineralized cortical bone particles, demineralized cortical bone fibers, granules or particles of non-demineralized cancellous bone, non-demineralized, cortical bone, or a combination of both, to produce graft material. Again, when implanted, such grafts are expected to provide bioactivity comparable to or the same as cancellous bone samples prior to devitalizing and other processing.

It is noted that the tissue-derived compositions need not be granules, particles, fibers or other physical forms comprising a plurality of smaller pieces. Rather, monolithic, or multi-component grafts having larger three-dimensional forms, such as bone-derived spinal bone spacers, bone-derived plugs, bone-derived struts, bone-derived pins, etc., may also be produced according to the methods described and contemplated herein to provide bone-derived grafts having three-dimensional forms. In some embodiments, the tissue-derived composition may have smaller physical forms, e.g., pieces or components, which are assembled, attached, or otherwise combined, to provide a larger three-dimensional shape by, for example without limitation, configuring the pieces or components having mating or interlocking features, shapes, contours, or combinations thereof, which facilitate temporary or permanent combination into the larger three-dimensional shape. As will be readily understood and within the ability of persons of ordinary skill in the relevant art, any one or more resizing steps may be performed, including but not limited to cutting, slicing, blending, shaving, milling, grinding, etc., to provide tissue-derived compositions in any of several physical forms, some but not all of which are discussed herein.

Moreover, it is contemplated that the tissue-derived compositions may be moldable, shapable, and reshapable, including after combination with a biocompatible material, before dehydration (e.g., prior to lyophilizing or another dehydration technique), after dehydration and subsequent rehydration, or some combination thereof. Furthermore, the tissue-derived compositions may be susceptible to molding, shaping, and reshaping, with or without using a mold or shaping device, or by manual manipulation (with or without an instrument or tool), as well as and before or during use. It is also contemplated that the tissue-derived compositions may be shaped or reshaped to have any desired three-dimensional shape (geometric, symmetric, asymmetric, irregular, and combinations thereof) which is retained thereafter, and for some time after implanting or administration, or until intentionally reshaped (partially or wholly) into a different desired three dimensional shape.

In some embodiments, for example without limitation, a devitalized cancellous bone tissue-derived composition may comprise a population of devitalized endogenous cells and their components, including endogenous extracellular vesicles, which have been retained in substantially undamaged condition from cancellous bone tissue samples to produce the composition. The retained devitalized endogenous cells do not exhibit metabolic activity. Such devitalized endogenous cells may carry bioavailable, bioactive substances, such as extracellular vesicles and their contents, expected to promote or facilitate the bone healing process, including the production of new bone tissue in-vivo, as well as promote angiogenic and/or osteoimmunomodulatory (OIM) potential in a recipient.

As used herein, the term “bioactive” means a substance or material which has a physiological effect on a recipient of the bioactive substance, where the physiological effect may be an intended component of treating a condition of the recipient.

As used herein, the term “bioactivity” includes physiological effects of a material, substance, or combination thereof, including, but not limited to, one or more of inducing, participating in, facilitating, or otherwise enhancing biological processes such as, without limitation, healing, generating, regenerating, repairing and reconstructing tissue, an organ, or a body feature, affected by a condition to be treated.

“Beneficial” bioactivity, as used herein, means one or more physiological effects which promote or increase the likelihood of achieving the intended goals of treatment, regardless of whether or not side effects or adverse effects may also be caused, promoted or facilitated. In other words, whether a bioactivity is “beneficial” will depend upon the goals of treatment, the degree to which one or more physiological effects are caused, promoted, or enhanced as compared to the degree or impact of any side effects or adverse effects which may also occur.

The term “biologically compatible” or “biocompatible” are used herein interchangeably to mean any material or substance (liquid, solid, particulate, solution, gel, etc.) which, when contacted with, administered to, or implanted on or in host (recipient) tissue, does not cause unacceptable levels of one or more adverse effects such as, without limitation, toxicity, injury, immune response (e.g., foreign body reaction or rejection, or irritation, allergic or other histamine reaction), disruption of cellular structure or function, etc.

The term “derived” is used herein to describe circumstances in which a material or substance has been made from an original or intermediate material, tissue, or substance, for example, without limitation, through physical processing, chemical processing, or a combination thereof. The aforesaid processing may involve one, two, or even several steps or phases, as well as repeated and alternating steps or phases. For example, processing steps may include subjecting one or more tissue samples to one or more of: separation, isolation, size reduction, decellularization, rinsing, disinfection, more rinsing, dehydration, and mixing, attaching, or otherwise combining with other materials, substances, components and structures.

As used herein, the terms “frozen” and “freeze” refer to exposing and holding a donor or tissue samples harvested therefrom at one or more temperatures below zero, and typically much lower than zero, such as about −10° C. and lower. Where a donor or tissue samples harvested therefrom are transported, shipped, or stored on wet ice, as is commonly performed in practice, for purposes of the invention described and contemplated herein, the donor or tissue samples would not be “frozen” and such wet ice treatment does not constitute a devitalizing pre-processing freezing step or freeze/thaw cycle.

As used herein, the terms “room temperature” and “ambient temperature” are used interchangeably to mean any one or more temperatures above freezing and above typical refrigeration temperatures (e.g., from about greater than 0° C. to about 10° C.), for example from greater than 0° C. to about 40° C., and more typically, but not limited to, from greater than about 18° C. to about 30° C.

As used herein, the terms “endogenous” and “native” are used interchangeably to describe components, materials, and substances (e.g., cells, cellular components, extracellular matrix and its structural matrix proteins, growth factors, exosomes, etc.), which were endogenous to and present in at least one tissue sample which was processed to produce the tissue-derived composition.

As used herein, the terms “resident” refers to cells which were originally present (i.e., endogenous and native to) and may also have been adherent (i.e., embedded, adhered, attached, or some combination thereof, either in, on, or both) to the extracellular matrix of at least one tissue sample which was processed to produce a tissue-derived composition and which remain present in the extracellular matrix of the tissue-derived composition. Endogenous cells need not be adherent and if they are adherent, they are not required to be embedded, adhered, attached, or some combination thereof, either in, on, or both, at exactly the same position or to the same degree in the extracellular matrix of the tissue-derived composition as they were in the starting tissue sample(s) to be considered retained and “resident.”

In some embodiments, the tissue-derived compositions have retained at least 10%, by weight (wt %), for example from about 10 to about 100 wt %, and any value therebetween, of endogenous cellular components based on the total weight of endogenous cellular components present in unprocessed tissue samples which were processed to produce the tissue-derived compositions. In some embodiments, the tissue-derived compositions have retained at least 25 wt % of endogenous cellular components based on the total weight of endogenous cellular components present in the unprocessed tissue samples. In some embodiments, the tissue-derived compositions have retained at least 50 wt % of endogenous cellular components based on the total weight of endogenous cellular components present in the unprocessed tissue samples. In some embodiments, the tissue-derived compositions have retained at least 90 wt % of endogenous cellular components based on the total weight of endogenous cellular components present in the unprocessed tissue samples. In some embodiments, the tissue-derived compositions have at retained least 95 wt % of endogenous cellular components based on the total weight of endogenous cellular components present in the unprocessed tissue samples. In some embodiments, the tissue-derived compositions have retained at least 98 wt % of endogenous cellular components based on the total weight of endogenous cellular components present in the unprocessed tissue samples. In some embodiments, the tissue-derived compositions have retained at least 99 wt % of endogenous cellular components based on the total weight of endogenous cellular components present in the unprocessed tissue samples.

In some embodiments, the tissue-derived compositions have retained at least 10 wt %, for example from about 10 to about 100 wt %, and any value therebetween, of endogenous cellular components relative to the total weight of endogenous cellular components present in milled and rinsed cancellous tissue sample(s) recovered from donors and processed within 72 hours post-mortem. In some embodiments, the tissue-derived compositions have retained at least 25 wt % of endogenous cellular components relative to the total weight of endogenous cellular components present in milled and rinsed cancellous tissue sample(s) recovered from donors and processed within 72 hours post-mortem. In some embodiments, the tissue-derived compositions have retained at least 50 wt % of endogenous cellular components relative to the total weight of endogenous cellular components present in milled and rinsed cancellous tissue sample(s) recovered from donors and processed within 72 hours post-mortem. In some embodiments, the tissue-derived compositions have retained at least 90 wt % of endogenous cellular components relative to the total weight of endogenous cellular components present in milled and rinsed cancellous tissue sample(s) recovered from donors and processed within 72 hours post-mortem. In some embodiments, the tissue-derived compositions have at retained least 95 wt % of endogenous cellular components relative to the total weight of endogenous cellular components present in milled and rinsed cancellous tissue sample(s) recovered from donors and processed within 72 hours post-mortem. In some embodiments, the tissue-derived compositions have retained at least 98 wt % of endogenous cellular components relative to the total weight of endogenous cellular components present in milled and rinsed cancellous tissue sample(s) recovered from donors and processed within 72 hours post-mortem. In some embodiments, the tissue-derived compositions have retained at least 99 wt % of endogenous cellular components relative to the total weight of endogenous cellular components present in milled and rinsed cancellous tissue sample(s) recovered from donors and processed within 72 hours post-mortem.

In some embodiments, the tissue-derived compositions have retained at least 10 wt % of endogenous cellular components based on the total weight of endogenous cellular components contained in viable cryopreserved tissue of the same tissue type and from the same donor (i.e., donor-matched) as the tissue-derived compositions. In some embodiments, the tissue-derived compositions have retained at least 25 wt % of endogenous cellular components based on the total weight of endogenous cellular components contained in donor-matched viable cryopreserved tissue. In some embodiments, the tissue-derived compositions have retained at least 50 wt % of endogenous cellular components based on the total weight of endogenous cellular components contained in donor matched viable cryopreserved tissue. In some embodiments, the tissue-derived compositions have retained at least 90 wt % of endogenous cellular components based on the total weight of endogenous cellular components contained in donor matched viable cryopreserved tissue. In some embodiments, the tissue-derived compositions have retained at least 95 wt % of endogenous cell-derived based on the total weight of endogenous cellular components contained in donor matched viable cryopreserved tissue. In some embodiments, the tissue-derived compositions have retained at least 98 wt % of endogenous cellular components based on the total weight of endogenous cellular components contained in donor matched viable cryopreserved tissue. In some embodiments, the tissue-derived compositions have retained at least 99 wt % of endogenous cellular components based on the total weight of endogenous cellular components contained in donor-matched viable cryopreserved tissue.

In some embodiments, the tissue-derived compositions have retained at least a minimum value of endogenous cellular components including but not limited to exosomes, as measured by the presence of CD63 antigen protein (CD63). In some embodiments, the tissue-derived composition has retained at least 10 picograms of CD63 per gram of the tissue-derived composition. In some embodiments, the tissue-derived composition has retained at least 50 picograms of CD63 per gram of the tissue-derived composition. In some embodiments, the tissue-derived composition has retained at least 100 picograms of CD63 per gram of the tissue-derived composition. In some embodiments, the tissue-derived composition has retained at least 500 picograms of CD63 per gram of the tissue-derived composition. In some embodiments, the tissue-derived composition has retained at least 1000 picograms of CD63 per gram of the tissue-derived composition. In some embodiments, the tissue-derived composition has retained at least 5000 picograms of CD63 per gram of the tissue-derived composition.

As used herein, the term “devitalized” describes one or more endogenous cells which remain resident in the retained ECM of the tissue-derived compositions and which are substantially incapable of metabolic activity prior to being implanted in a recipient. “Devitalized” cells are generally substantially undamaged and undisrupted (i.e., not lysed or having an otherwise open cellular membrane) and contain cellular components including, without limitation, exosomes, DNA, etc., yet lack measurable metabolic activity. Upon disruption of devitalized cells, cellular membranes are lysed and cellular components, such as exosomes and other bioactive substances, may be liberated into the ECM of the tissue-derived compositions and, since they are unbound, be bioavailable to a recipient of a devitalized tissue-derived composition.

Subsequently, the number of cells that exhibit positive staining for select markers can be counted by using automated image analysis algorithms that have been validated to only detect cells that exhibit a positive stain. Many different markers could be used to identify the types of bone-forming cells present in the bone. For example, CD166, a cell surface marker indicative of MSCs, can be determined along with the number of cells that have positive staining for Osterix, a protein that indicates the presence of osteoprogenitor cells. Other markers could include osteocalcin, a protein that indicates the presence of osteoblasts and other bone-forming cells. If counting more than one population of cells, correction factors may need to be established to ensure that cells are not mistakenly double counted if a given cell type could express more than one marker that is being evaluated. In order to obtain a concentration of cells/cc, multiple 2D histological sections can be stacked together and cells can be counted to determine an estimate of cells per unit volume using stereological methods.

A significant number of cells are expected to remain attached to the bone matrix (ECM) following processing to produce the devitalized bone-derived compositions from bone samples (as well as following and rehydration or other reconstitution). In some embodiments, the devitalized allograft bone derived compositions may contain at least 100 devitalized cells per cubic centimeter of devitalized bone-derived compositions, when cells positive for at least one of the following markers are counted: Hematoxylin and Eosin (H&E), CD166, Osterix and osteocalcin. In some embodiments, the devitalized allograft bone derived compositions may have a minimum concentration of devitalized endogenous cells in a range of from about 5000 cells per cubic centimeter (cc) of the devitalized allograft bone derived compositions to about 2 million cells/cc of the devitalized allograft bone derived compositions.

Devitalized tissue-derived compositions, such as devitalized cancellous bone-derived compositions, which are useful as graft material will now be described in more detail. Devitalized cancellous bone-derived compositions contain devitalized endogenous cells which remain resident in, on, or both, the extracellular matrix of the cancellous bone-derived compositions. Additionally, the cancellous bone-derived compositions may be exposed to or contacted with one or more preservatives, in solution or not, and then lyophilized. Lyophilizing may be performed without any preservatives. In lyophilized form, the devitalized cancellous bone-derived compositions may be rehydrated with a biocompatible carrier or liquid prior to or at the time of use (implantation). These compositions would not need to be thawed prior to use (implantation), which saves time during medical procedures. The devitalized cancellous bone-derived compositions provide increased beneficial bioactivity compared to decellularized cancellous bone-derived compositions, since the devitalized cancellous bone-derived compositions still contain one or more endogenous bioactive substances within the undisrupted devitalized endogenous cells, in addition to any extracellular vesicles and other beneficial substances that may be present in the ECM, but outside the devitalized cells. Devitalized cells may induce efferocytosis, a process by which host immune cells, typically macrophages, engulf and digest dead or dying cells, clearing them from tissues and producing an anti-inflammatory effect. Decellularized compositions have lost the cells as well as at least some of the extracellular vesicles and other bioactive substances which were contained in the cells or in the ECM.

The devitalized cancellous bone-derived compositions described and contemplated herein: (1) provide an allograft formulation comparable or improved compared to known mineralized and demineralized bone matrix (DBM) materials and mineralized and demineralized bone fibers (DBFs), allogenic as well as autologous, (2) deliver desirable handling properties (e.g., moldability, cohesiveness, irrigation-resistance, shape retention upon rehydration, etc.) which are similar to known bone-derived compositions (e.g., graft material comprising a combination of cancellous bone with viable cells retained and resident within the extracellular matrix thereof and demineralized cortical bone pieces, granules, fibers, or particulates as well as bone graft material containing viable cells and bioactive components thereof that are beneficial for bone healing), (3) allow for convenient room temperature or other ambient temperature storage by end users, and (4) eliminates thawing time (otherwise necessary when using cryopreserved or frozen bone-derived graft materials).

The novelty and advantageousness of the devitalized bone-derived compositions, whether derived from allogenic, autogenic, or xenogenic bone tissue samples, and the processing methods for producing them, relate to the ability to retain and maintain any one or more resident endogenous exosomes, bioactive factors, and non-viable (devitalized) cells in the bone-derived extracellular matrix, on the bone extracellular matrix, or both, following processing, and the ability to be stored at ambient temperature. While the processing methods disclosed and contemplated herein do not retain the viability of endogenous cells, they do retain elevated levels of proteins, extracellular vesicles/exosomes, and angiogenic, osteogenic, and OIM potential in the devitalized bone-derived compositions. Furthermore, in some embodiments, endogenous bioactive substances, such as one or more of exosomes and growth factors, may remain resident and bioactive in the devitalized bone-derived compositions, independently and even in the absence of the presence of non-viable endogenous cells.

In an exemplary embodiment, a tissue-derived composition is provided which is comprised of devitalized bone tissue with a population of retained devitalized (non-viable) endogenous cells and endogenous cellular components, including extracellular vesicles, is preserved with biocompatible components, and has the following characteristics:
Retained endogenous exosomes that contain either proteins or transcriptome that can impart a positive healing effect in bone,
Retained non-viable cells that provide higher levels of proteins and growth factors compared to non-cellular counterparts, and
Retained non-viable cells and/or bioactive components that can impart an osteoimmunomodulatory effect.

In an exemplary embodiment, a method is provided for making a devitalized bone tissue-derived graft with retained extracellular vesicles. Characterization methods are described which demonstrate the retention of extracellular vesicles, such as exosomes, indirectly by identifying specific markers, including CD-9, CD-63, and CD-81 expressed on the surface of retained endogenous exosomes, and TSG101 and HSP70 expressed internally within exosomes. Exosomes can also be visualized through different imaging modalities including transmission electronic microscopy (TEM), scanning electron microscopy (SEM), Cryo-electron microscopy, and nanoparticle tracking analysis (NTA).

Most bone-derived grafts are decellularized during processing as a result of exposure to traditional cleaning, disinfection agents, demineralization reagents, and combinations thereof. Other bone-derived grafts, such as those more gently processed to preserve the cells, are generally designed to maintain cell viability, and are most commonly cryopreserved and stored frozen in their final forms. While viable cell containing bone allografts have benefits compared to standard demineralized or non-demineralized bone allografts, donors and tissue samples harvested therefrom cannot be frozen prior to processing and must be processed “at risk” to produce viable cell containing bone allografts, meaning that processing is initiated prior to obtaining bioburden test results, since there is not sufficient time to complete the testing before processing must commence to provide the best opportunity for maintaining cell viability.

The presence of retained devitalized endogenous cells in the tissue-derived compositions enable retention of more of the endogenous bioactive substances from the original tissue samples than are typically retained in in other tissue-derived compositions because the retained devitalized endogenous cells contain native bioactive substances which are often otherwise lost during decellularization processes. Upon disruption of the retained devitalized endogenous cells, the bioactive substances held therein may be liberated into the ECM of the tissue-derived compositions and grafts comprising them. Such bioactive substances liberated from retained disrupted devitalized native cells include proteins, exosomes, growth factors, and cytokines that are often otherwise lost when tissue-derived graft matrices have been decellularized.

Additionally, but without wishing to be bound by theory, the process by which host macrophages engulf a cellular corpse, known as efferocytosis, may play a role in creating a favorable osteoimmunmodulatory environment by inducing the polarization of M2 macrophages, which are the cells that perform these actions. Thus, the devitalized cells in the devitalized bone derived composition may also elicit such a response in the patient, thereby contributing to a more pro-healing environment. Again, without wishing to be bound by theory, it is believed that such favorable osteoimmunomodulation may serve to recalibrate dysregulated inflammation in compromised, hard-to-heal patients, whereby the devitalized tissue-derived compositions would be more effective for bone repair and regeneration compared to standard bone allografts.

The production of bone-derived compositions and grafts, as described and contemplated herein, is accomplished by performing method steps and techniques carefully selected and designed to retain native cells and/or bioactive substances present (in, on, or both) the extracellular matrix of tissue samples throughout processing. Where native cells are retained, the method steps and techniques are selected and intended to devitalize those retained native cells throughout processing, so that they remain in the tissue-derived compositions in devitalized and undisrupted condition. Methods for producing the devitalizing native cells may, for example without limitation, comprise performing at least one freeze-thaw cycle or step, exposing the cells to reagents that may disrupt metabolic activity that can include degrading or destroying at least a portion of ATP present in the tissue samples (e.g., contacting with an enzyme or incubating in a biologically compatible fluid to hydrolyze ATP), performing a pre- or post-treatment irradiation step, or a combination thereof.

In particular, interruption and cessation of cellular metabolic activity may be augmented by use of one or more enzymes which degrade, eliminate, or both, some or all adenosine triphosphate (ATP), such as, but not limited to, by hydrolyzing ATP. The resulting decreased amounts, or absence, of ATP, in turn, inhibits the metabolic functionality of cells present in the tissue sample(s). One such enzyme, for example without limitation, is apyrase which catalyzes the hydrolysis of ATP and/or adenosine diphosphate (ADP) into adenosine monophosphate (AMP). Regardless of how and when the devitalizing is accomplished, the chemicals used to treat and process the bone tissue sample(s) are intended to clean the bone while retaining undisrupted endogenous cells and cell-matrix interactions and attachments. In particular, direct exposure to detergents, alcohols, acids and oxidizing agents should be kept to a minimum, involve appropriately low but effective concentrations, or altogether eliminated, during the production process.

Additionally, if lyophilization is performed as part of the processing to produce the devitalized bone tissue-derived compositions, it should also be performed in a manner designed and expected to best maintain cellular components and cell-matrix interactions and attachments initially present in the bone tissue sample. This may involve the inclusion of one or more preservatives (optionally, in an aqueous solution) which are contacted with bone tissue samples.

Freezing steps, irradiation steps, or both are selected, designed and performed in a way that devitalizes the bioactive native cells of the bone tissue sample(s), while retaining a significant portion of the devitalized cells resident, undisrupted, and adherent in or to (or both) the extracellular matrix.

As will be recognized and determinable by persons of ordinary skill in the relevant art based on the present disclosure and descriptions of exemplary embodiments, the type of tissue in the one or more tissue samples being processed should be a factor in selecting the method steps and techniques (such as up-front freezing, freeze-thaw cycles, cleaning, disinfecting, lyophilizing, sterilizing, etc.), to be applied to the tissue sample.

Similarly, several characteristics of the one or more tissue samples being processed can be factors to be considered when selecting the types and concentrations of the chemicals and other substances (e.g., detergents, alcohols, acids and oxidizing agents) used in the various production method steps and techniques, to be applied to the tissue sample(s). Such tissue characteristics may include one or more of: tissue type, tissue porosity, density, size and/or dimensions, composition (e.g., what types and proportions of ECM proteins are present, such as types of collagen, fibronectin, etc., what types of cells are present, etc.). For example, while processing techniques applied to tissue samples to produce the tissue-derived compositions should be gentle regardless of tissue type, bone samples may generally be effectively contacted with or exposed to harsher disinfecting agents or detergents, higher concentrations of them or longer durations of time, than softer tissue types such as adipose or placenta tissue samples (which are less dense and are relatively free of calcium-containing compounds compared to bone). In some embodiments, no detergents or oxidizing agents are contacted with the tissue samples during processing to produce the tissue-derived compositions.

Following processing and lyophilization, a devitalized bone-derived allograft composition retains a population of devitalized native cells that can be visualized in the matrix using histological staining/imaging methods but will exhibit minimal to no metabolic cell activity as tested by the CellTiterGlo® cell viability assay. This assay is based on the quantification of ATP which is present in all metabolically active cells and is an indicator of cell viability.

In some embodiments, “minimal to no metabolic activity,” as used herein to describe the devitalized tissue-derived composition, means from about 30% to about 0%, or any value therebetween, such as without limitation, less than about 25%, or less than about 20%, or less than about 15%, or less than about 10%, or less than about 8%, or less than about 5%, or less than about 3%, or less than about 1% of metabolic activity by endogenous cells present in unprocessed bone tissue sample(s) of the same tissue type as the devitalized tissue-derived composition.

In some embodiments, “minimal to no metabolic activity,” as used herein to describe the devitalized tissue-derived composition, means from about 40% to about 0%, or any value therebetween, such as without limitation, less than about 35%, or less than about 30%, less than about 25%, or less than about 20%, or less than about 15%, or less than about 10%, or less than about 8%, or less than about 5%, or less than about 3%, or less than about 1%, of metabolic activity by endogenous cells present in milled and rinsed cancellous bone sample(s) recovered from donors and processed within 72 hours post-mortem.

In some embodiments, “minimal to no metabolic activity,” as used herein to describe the devitalized tissue-derived composition, means from about 50% to about 0%, or any value therebetween, such as without limitation, less than about 45%, or less than about 40%, or less than about 35%, or less than about 30%, or less than about 25%, or less than about 20%, or less than about 15%, or less than about 10%, or less than about 8%, or less than about 5%, or less than about 3%, or less than about 1%, of metabolic activity by endogenous cells present in viable cryopreserved bone tissue.

In some embodiments, “minimal to no metabolic activity,” as used herein to describe the devitalized tissue-derived composition, means from about 0.075 nanomoles (nmol) to about 0 nmol ATP per gram of the devitalized tissue-derived composition, or any value therebetween, such as without limitation, less than about 0.07 nmol ATP/g, or less than about 0.06 nmol ATP/g, or less than about 0.05 nmol ATP/g, or less than about 0.04 nmol ATP, or less than about 0.03 nmol ATP, or less than about 0.02 nmol ATP, or less than about 0.01 nmol ATP, per gram of the devitalized tissue-derived composition. Due to the presence of the non-viable (devitalized) endogenous cells and minimal processing, increased levels of proteins, growth factors, extracellular vesicles, and/or cytokines are expected to be present compared to standard bone allografts derived from the same donors.

Generally, some embodiments of the methods for producing the devitalized tissue-derived composition include a pre-treatment step comprising devitalizing native cells present in tissue samples to be processed. In some embodiments, the methods also comprise a lyophilizing step which may or may not include contacting the tissue samples with one or more preservatives, where the preservatives are selected and combined so as to minimize or avoid maintenance of cell viability.

In exemplary methods for producing the devitalized tissue-derived compositions, donors and tissue samples therefrom may first be handled and managed without specific attention to preserve the viability of the native cells, as is a concern with donor tissue samples intended for use to produce viable tissue-derived matrices. Rather, tissue samples can be frozen and thawed, at least once, to retain the endogenous cellular components in the tissue matrix without preserving native cell viability. In some embodiments, the tissue samples may be subjected to two or more freeze/thaw cycles to accomplish devitalizing of cells while retaining them in the tissue matrix. Alternatively, the tissue samples may be irradiated with gamma rays, x-rays, or e-beam, exposed to ultraviolet light, or even exposed to heat or ethylene oxide, as well as other disinfecting and sterilizing techniques, and combinations thereof, as long as cellular components are sufficiently maintained (e.g., not lysed) including extracellular vesicles and other beneficial cell-associated substances.

The presence and retention of devitalized native cells in devitalized tissue-derived compositions, such as devitalized cancellous bone-derived compositions, is a feature of the invention described and contemplated herein. Cancellous bone samples used to produce such devitalized tissue-derived compositions and graft compositions including them are typically, but not necessarily, recovered from the hemi-pelvis, femur, tibia, talus, vertebrae, calcaneus, humerus, sternum or other cancellous bone sites of a donor that contain viable bone-forming cells or mesenchymal stem cells. Prior to processing, the donor and/or tissue sample is subjected to at least one pretreatment devitalizing technique. At least one, such as two or more, devitalizing pre-treatment freeze/thaw cycle may be performed to devitalize (i.e., reduce or eliminate the viability of) the native cells present in the tissue samples. Such devitalizing pre-treatment may alternatively be performed using irradiation, such as gamma or E-beam irradiation, or ultraviolet light exposure, to devitalize the native cells. Devitalization may also be accomplished by use of reagents or enzymes which disrupt metabolic activity (e.g., catalyze the degradation or destruction of ATP, such as apyrase), optionally followed incubation in biologically compatible fluid (e.g. saline, PBS) to hydrolyze remaining ATP or metabolically functioning components to render the tissue devitalized (i.e., at least in part by inhibiting metabolic activity of retained native cells). In some embodiments, a combination of two or more of the aforesaid devitalizing techniques may be performed.

Generally, after cleaning and debridement to isolate the cancellous tissue, the cancellous tissue may then be resized by cutting into blocks and then milled to produce cancellous granules. Milling may, for example without limitation, produce cancellous particles having a population in a size range of from about 20 microns (um) to about 6 millimeters (mm), as well as any range and values therebetween, such as from about 20 μm to about 4 mm, or from about 20 μm to about 2 mm, or from about 40 to about 1 mm, or from about 50 μm to about 2 μm, or from about 200 μm to about 2 mm.

Milled cancellous granules may be placed in a preservative media to retain undisrupted endogenous cells while other processing tasks are performed. Once all mineralized cancellous granule components are ready, the granules are aliquoted into primary storage containers (with or without a preservative), and the tissue granules are dehydrated. Dehydrating may, for example without limitation, be performed by lyophilizing, with or without a preservative. In some embodiments, other allogeneic bone components such as demineralized bone fibers may be mixed or otherwise combined with the granules, and the combination then aliquoted into primary storage containers.

An exemplary method for producing devitalized tissue-derived compositions from one or more cancellous bone samples will now be described and includes the steps of:
(1) devitalizing native cells of a cancellous bone sample or a tissue-derived composition produced therefrom by one or more of the following techniques:
   a. prior to any processing steps, either:
      (i) subjecting the cancellous bone sample to at least one freeze/thaw cycle, each of which comprises freezing the cancellous bone sample and then at least partially thawing the frozen cancellous bone sample, wherein if the cancellous bone sample is previously frozen, then devitalizing is performed by at least partially thawing the frozen cancellous bone sample at least once, with or without additional freeze/thaw cycles, wherein the freeze-thaw cycles are preferably performed under conditions which minimize or avoid lysis or other damage to cellular membranes; or
      (ii) subjecting the cancellous bone sample to irradiation; or
      (iii) performing a combination of both (i) and (ii); and
   b. direct processing of milled cancellous bone, either:
      (i) subjecting the cancellous bone to a reagent or an enzyme that disrupts metabolic activity such as apyrase (i.e., which catalyzes the degradation or destruction of ATP (e.g., by hydrolysis)), or;
      (ii) incubating processed cancellous bone in biologically compatible fluid to hydrolyze retained metabolic components; or
      (iii) performing a combination of (i) and (ii) and
   c. optionally, after all processing steps have been performed, subjecting a cancellous bone-derived composition produced from the cancellous bone sample to terminal sterilization by exposing the cancellous bone-derived composition to radiation or ethylene oxide;
(2) optionally, obtaining and testing a specimen of the cancellous bone sample and determining the bioburden level of the cancellous bone sample;
(3) isolating the cancellous bone sample by cutting, debriding, etc., to remove soft tissue, muscle, and fat attachments;
(4) rising the cancellous bone sample to remove bioburden, blood and lipids;
(5) debriding the cancellous bone sample to remove additional soft tissue;
(6) cutting the cancellous bone sample into smaller pieces, such as blocks;
(7) milling cancellous bone sample pieces into cancellous bone granules;
   a. optionally, rinse cancellous bone granules with a biocompatible liquid (e.g., PBS);
   b. devitalizing cancellous tissue using enzymes, hydrolyzing incubation steps, or a combination of both;
(8) Optionally, obtain or produce demineralized cortical bone fibers (e.g., cut cortical bone sample, mill cortical bone sample into cortical bone fibers, demineralize cortical fibers, and rinse demineralized cortical bone fibers), and mix demineralized cortical bone fibers with rinsed cancellous bone granules in a desired predetermined ratio;
(9) optionally, contacting the cortical bone granules with a preservative, for a contacting period of time;
(10) lyophilizing cancellous bone granules;
(11) optionally, after lyophilizing, obtaining and testing a specimen of the cancellous bone sample and determining the bioburden level of the cancellous bone sample;
(12) storing the lyophilized cancellous bone granules for a storing period of time, at one or more temperatures above freezing, such as refrigeration temperatures (i.e., from greater than 0° C. to about 10° C.) or ambient/room temperatures (i.e., from greater than 10° C. to about 40° C.); and
   a. lyophilized cancellous bone granules, with or without additional tissue-derived or other materials, may be stored in a sealed, sterile container at one or more temperatures above freezing (e.g., refrigerated, ambient or room temperatures).
(13) optionally, prior to or at the time of use, rehydrating the cancellous bone granules with a biocompatible carrier, such as without limitation, saline, PBS, blood, plasma, bone marrow, aspirate, PRP, SVF or other cell containing solution, one or more antibiotic agents (in solution or not), and comparable solutions.

In some embodiments, demineralized, partially demineralized or non-demineralized cortical bone derived matrices may be mixed or otherwise combined with the cancellous bone granules to produce a composite devitalized bone tissue-derived composition suitable for use as graft material. To produce one or more cortical bone derived matrices, one or more cortical bone samples would be stripped of soft tissue and cancellous bone, optionally demineralized and modified geometrically, either pre- or post-demineralization, to a configuration with desired handling properties. Suitable cortical bone samples may be recovered, for example without limitation, from the femur, tibia, humerus, radius, ulna, and fibula, or other suitable long bones of a donor. Geometric (i.e., size, shape, or both) modification may produce any of several suitable physical forms including, but not limited to, fibers, pieces, particulates such as granules and powder, and combinations thereof.

For example, in some such embodiments, the cortical bone-derived matrix may comprise demineralized cortical bone fibers (DBFs), which are produced from cortical bone samples. In such embodiments, the long bones would first be stripped of soft tissue and the shaft cores would be cleared of nearly all of the cancellous bone. The cortical shafts may be cleaned using detergents and/or surfactants to remove residual blood and lipids, then cut into cross-sectional segments of the appropriate length for milling. For example, without limitation, milling of the shaft cross-sections produces elongated fibers of cortical bone, which may then be demineralized in dilute acid to provide DBFs for combination with cancellous bone granules in desired proportions, with or without the addition of storage media, a buffered carrier, or both. The composite devitalized bone tissue-derived composition may then be aliquoted into primary storage containers, with or without preservatives, and lyophilized.

In one exemplary embodiment, a graft composition comprises a mixture of a mineralized devitalized cancellous bone-derived composition and a demineralized cortical bone-derived composition. The mineralized devitalized cancellous bone-derived composition is produced according to an embodiment of the present invention and, therefore, retains one or more of: endogenous devitalized (non-viable) cells, and cellular components, including extracellular vesicles such as exosomes. The demineralized cortical bone-derived composition (or matrix) may include any one or more of several possible geometries (i.e., physical shapes), including without limitation. fibers of varying lengths, fibers of varying thicknesses, granules of varying diameters, and combinations thereof. The aforesaid composite graft composition which comprises the devitalized cancellous bone-derived composition is useful, for example without limitation, for use as a bone void filler or a bone graft extender, for the treatment of musculoskeletal defects.

Additionally, such composite graft compositions may further comprise one or more other materials or compositions including, but not limited to, processed bone marrow, endosteum, periosteum, etc. The final composite graft composition may be preserved at ambient temperature via lyophilization with one or more suitable preservatives as previously described and listed above.

An exemplary method for producing the aforesaid composite graft composition comprising a mixture of devitalized cancellous bone-derived composition and cortical bone-derived composition will now be described. This exemplary method generally includes making a devitalized cancellous bone tissue-derived composition from one or more cancellous bone tissue samples and combining the composition with a cortical bone tissue-derived composition, to provide the graft composition having the properties and benefits of the devitalized bone tissue-derived composition. In some further embodiments, the method also includes making the cortical bone tissue-derived composition from one or more cortical bone tissue sample, and then combining it with the devitalized cancellous bone tissue-derived composition.

Cancellous bone and cortical bone anatomical sites are separated from the donor's other anatomical sites and tissues (i.e., recovered). The anatomical sites from which cancellous bone tissue samples are recovered are not limited, but may, without limitation, include: pelvis (specifically hemi pelvis), femur, humerus, talus, calcaneus, tibia and vertebral body. The anatomical sites from which cortical bone tissue samples are recovered are not limited, but may, without limitation, include the long bones of the femur, tibia, humerus, ulna, fibula and radius.

After recovering is completed, both cortical and cancellous bone tissue samples undergo debridement and a series of cleaning rinses. Afterwards, at least one cancellous tissue sample is cut into cancellous blocks and may, optionally, be staged (i.e., stored) in biologically compatible fluid. The cancellous blocks are milled to fine granules, ranging in various sizes which may also, optionally, be staged in biologically compatible fluid. The cancellous granules are then rinsed in PBS, followed by rinsing in dilute acetic acid (e.g., without limitation, 0.1N), with two more subsequent PBS rinse steps. Cancellous granules may also be exposed to enzymes such as apyrase for up to 60 mins, as previously described, for hydrolyzing and degrading ATP. Cancellous granules are then staged in biologically compatible fluid such as PBS for several hours prior to mixing with cortical bone.

On the other hand, the cortical tissue samples comprising long bone sections are cut to the desired length(s), the marrow removed therefrom (marrow may be retained for further downstream processing and retention, or not), and then the cut cortical tissue samples are delipidized. At least one cut cortical tissue sample is then further cut (e.g., grated, milled, shaved, etc.) into fibers such as (without limitation) using a lathe or end-mill cutter. Any residual cortical cross sections which were unable to be cut into fibers may, optionally, be milled further into cortical particles and, optionally, added to or combined with the cortical bone fibers. Cortical bone fibers are then demineralized in hydrochloric acid (HCl) solution and subjected to subsequent rinses and soaks of water and buffer solution.

The cancellous granules and cortical bone fibers are combined in various ratios, and desired quantities of the resulting mixture are aliquoted into 1 oz jars (or other containers suitable for holding quantities of the mixture). A quantity of a preservative solution (e.g., containing a disaccharide and saline) which is sufficient to cover the mixture of cancellous granules and cortical bone fibers in each jar is added to each jar. Each jar is then covered and lyophilized. The final lyophilized devitalized composition contains a first component comprising demineralized cortical bone fibers and a second component comprising mineralized cancellous granules which retain endogenous non-viable cells and cellular components, including extracellular vesicles. In some embodiments, the mineralized cancellous granules may retain either endogenous non-viable cells or one or more and cellular components, such as exosomes.

All processing is conducted in an aseptic manner. Processing is designed to remove immunoreactive elements and is demonstrated to not elicit an immune response as determined by performing an MLR assay. All selected preservation and protectant agents are biocompatible.

Sterility of the tissue graft may be assessed by following the USP <71> guidelines, or by utilizing a terminal sterilization technique. For terminally sterilized compositions, the preservative could also contain a free radical scavenger or other radio-protectant agent.

An exemplary devitalized cancellous bone-derived composition produced by the foregoing exemplary method may have the following composition and properties:

### I. Allograft Bone:

### Mineralized Devitalized Cancellous Bone Granules Containing Devitalized Endogenous Cells

Optionally, combine with:
Demineralized Cortical Bone Fibers in a proportion of about 30-50% Devitalized Cortical Bone Granules and about 50-70% Demineralized Cortical Bone Fibers, by weight, based on the total weight of both bone the cortical and cancellous bone components;

### II. Optionally, a Preservative, Whether in Solution or not.

Another exemplary devitalized cancellous bone-derived composition produced by the foregoing exemplary method may have the following composition and properties:

### I. Allograft Bone:

### Mineralized Devitalized Cancellous Bone Granules Containing Devitalized Endogenous Cells

Optionally, combine with:
Demineralized Cortical Bone Fibers, in a proportion of about 40-60% Devitalized Cortical Bone Granules and with the remainder being Demineralized Cortical Bone Fibers, by weight, based on the total weight of both bone the cortical and cancellous bone components;

### II. Optionally, Preservative, Whether in Solution or not.

As will be recognized by persons of ordinary skill in the relevant art, the devitalized cancellous bone-derived compositions are suitable for various uses, treatments, and applications. For example, but without limitation, it may be used as a surgical graft for the treatment of bone defects. Contemplated clinical applications for the devitalized cancellous bone-derived compositions include usage in orthopaedic surgery and other therapeutic treatments to promote fusion where needed, e.g., intervertebral spinal fusion, posterolateral spinal fusion, long bone fusion, foot-and-ankle fusion or anywhere bone fusion or bony defect filing is required or beneficial.

Additionally, it is contemplated and foreseeable that the presently described devitalized cancellous bone-derived compositions will be useful in any of several therapeutic treatments to address any of several bone-related conditions and issues. Such treatments and conditions include, but are not limited to: general indications involving bone defects and conditions, spinal applications, orthopedic trauma and reconstruction, extremity and small bone procedures, pelvic and acetabular applications, and craniofacial and oral/maxillofacial applications.

More particularly, the devitalized cancellous bone-derived compositions provide therapeutic advantages when used to treat general indications involving bone defects and conditions including without limitation: filling bony voids or gaps not intrinsic to structural stability, augmentation in nonunion or delayed union fractures, providing a bone graft extender in combination with autograft or bone marrow aspirate, and for any of various body features and conditions where bone fusion or bony defect filling is required or beneficial, including but not limited to defects associated with oncology, trauma, reconstruction, and elective procedures.

The devitalized cancellous bone-derived compositions also provide therapeutic advantages when used for spine-related applications and conditions, including without limitation: intervertebral spinal fusion (cervical, thoracic, lumbar), posterolateral spinal fusion, spinal deformity correction or modification (e.g., scoliosis, kyphosis, lordosis, etc.), and revision spinal fusion.

Conditions and treatments involving orthopedic trauma and reconstruction which may be effectively addressed using the devitalized cancellous bone-derived compositions include, but are not limited to: long bone fusion (e.g., femur, tibia, humerus, etc.), repair of traumatic bone defects or comminuted fractures, cavitary defects remaining after tumor resection or cyst removal, revision arthroplasty (e.g., of the hip, knee, shoulder, etc.).

The devitalized cancellous bone-derived compositions are also suitable for use in extremity and small bone procedures including, without limitation: foot and ankle fusion (e.g., hindfoot, midfoot, forefoot), hand and wrist fusion or reconstruction, and osteotomies requiring or otherwise involving bone graft augmentation.

Conditions and treatments involving pelvic and acetabular applications may also be effectively addressed using the devitalized cancellous bone-derived compositions and include, but are not limited to: pelvic reconstruction following trauma or tumor excision, and acetabular defect filling such as during hip revision surgery.

Additionally, the devitalized cancellous bone-derived compositions may also provide therapeutic benefits when used for treating craniofacial and oral/maxillofacial conditions during procedures such as, without limitation: bone loss restoration or/and bone volumization for plastic, reconstructive and aesthetic applications, craniofacial augmentation/reconstruction (e.g., congenital defect repair, Le Fort osteotomies, etc.), dental intraosseous defects (periodontal/infrabony defects), alveolar ridge augmentation (e.g., sinus lift, implant preparation, etc.), and cystic defect filling in oral surgery.

Current cell-based bone grafts are generally cryopreserved to maintain long-term cell viability (up to five years), requiring end users to store the grafts at approximately −70° C. or even −80° C. until use. This requires a thawing step prior to implantation and can also present some logistical issues for surgical centers that do not have the appropriate freezer equipment.

Room-temperature or refrigerated storage of devitalized bone tissue-derived compositions, which are useful as grafts, provides increased efficiencies for both end users and tissue processors. They provide improved ease-of-use for the user by eliminating the need to thaw before use and also offers added convenience to those users who are likely to use the graft shortly after receipt and/or lack the appropriate storage facilities for frozen tissue. For tissue processors, non-frozen storage eliminates the need for costly storage resources (dry ice, liquid nitrogen, tanks and freezers, etc.) and removes certain limitations on tissue shipment (limit on shipment time and size due to dry ice).

Furthermore, possible applications of the devitalized tissue-derived compositions, such as mineralized devitalized bone tissue-derived compositions include fusion of extremities including, without limitation, an ankle, a foot, a wrist, a hand, a finger, a toe, etc. It is contemplated that devitalized bone tissue-derived compositions would be useful and effective anywhere bone repair or fusion is needed, with the potential to provide additional benefits to hard-to-heal conditions and patients.

It will be understood that the embodiments of the present invention described hereinabove are merely exemplary and that a person skilled in the art may make variations and modifications without departing from the spirit and scope of the invention. All such variations and modifications are intended to be included within the scope of the present invention.

### EXAMPLES

The following examples are intended to provide those of ordinary skill in the relevant art with a complete and enabling disclosure and description of exemplary embodiments of the described invention, but are not intended to limit the scope of what the inventors regard as their invention, nor are the following examples intended to represent that the experiments below are all or the only experiments performed. Efforts have been made to ensure accuracy with respect to numbers used (e.g., amounts, temperatures, etc.) but some experimental errors and deviations should be accounted for. Unless indicated otherwise, parts are parts by weight, molecular weight is weight average molecular weight, temperature is in degrees centigrade (° C.), and pressure is at or near atmospheric. Although the exemplary embodiments are adapted to specific types of tissue, it will be recognized by those skilled in the relevant art that the methods described herein can be readily adapted to other types of tissue.

### Example 1

### Exosome Content Determined Based on CD63 Using Various Separation Techniques for Cryopreserved, Devitalized, and Decellularized Human Cancellous Bone Compositions

### Donor Processing:

Cadaver tissue was received, and the left side of the donor separated from the right. The left side was used for fresh-tissue groups and processed within 72-hours post-mortem in order to retain viable cellular component. The right side of the donor was devitalized by performing two freeze-thaw cycles to mimic standard frozen donor processing. Cancellous bone tissue was processed into blocks (i.e., by debriding, dissection, and soaking in basal media, as described in Example 3 below), with the positive control undergoing zero freeze-thaw cycles and both the devitalized and decellularized cancellous groups undergoing two freeze-thaw cycles. Cancellous blocks for all test groups were milled by conventional methods to cancellous bone particles in a range of from 212 μm to 2 mm. The positive control underwent PBS, acetic acid, and two more PBS rinses. The devitalized test group underwent PBS, acetic acid, PBS, apyrase, and a final PBS rinse. To remove the cells from the “Decellularized” group, milled cancellous tissue underwent chemical processing including a mild surfactant, alcohol, and an oxidizing agent.

### Tissue Groups & Descriptions:

Positive Control: fresh-donor derived (zero freeze-thaws) milled cancellous tissue cryopreserved in cell culture media and 10% DMSO
Devitalized Cancellous (Freeze/Thaw ×2): frozen-donor derived cancellous tissue lyophilized in 0.4M trehalose-saline solution
Decellularized Cancellous: frozen-donor derived and chemically processed (mild surfactant, alcohol, and an oxidizing agent) cancellous lyopreserved in 0.4M trehalose-saline solution

### Exosome Extraction:

All test groups underwent two enzymatic extraction methods:
   1. Collagenase—24 hr digestion in Collagenase II at 20 U/mL in DMEM F12 and 15 mM HEPES
   2. Collagenase & DNAse—24 hr digestion in Collagenase II at 20 U/mL and DNAse at 40 U/mL in DMEM F12 and 15 mM HEPES

### Exosome Isolation:

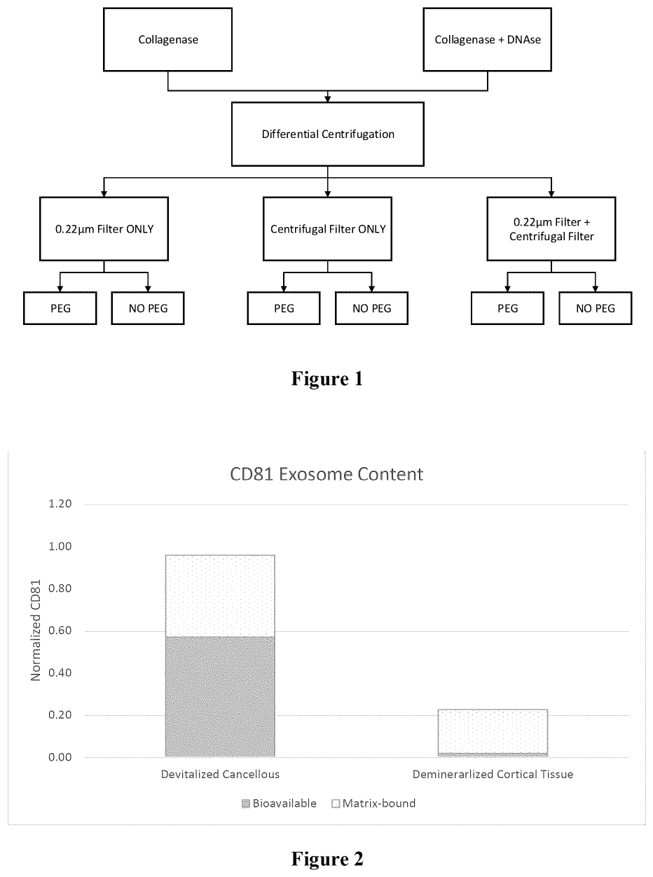

After 24 hrs were complete for all extraction methods, each test group underwent the following subsequent purification steps:
Differential Centrifugation:
   1. Centrifuge at 300 g for 10 minutes @4 C. Discard floating tissue and transfer supernatant to new 5 mL tubes.
   2. Centrifuge at 2000 g for 20 minutes @4 C. Transfer supernatant to new 5 mL tubes.
After differential centrifugation, test groups were either purified via 1) filtration through 0.22 um filter only, 2) centrifugal filtration only, or 3) filtration through a 0.22 um filter followed by centrifugal filtration. A detailed summary of purification steps is detailed below and test groups are outlined in FIG. 1.
Filtration:
   1. Filter supernatant with 0.22 μm pore size syringe filters (0.22 μm Filter groups ONLY).
      Centrifugal Filter groups should be dispensed directly from the syringe into the centrifugal filters.
   2. Use 4 mL AMICON filters (MWCO of 100 kDa) to centrifuge at 3000 g for 15 minutes @4 C (Centrifugal Filter groups ONLY).
      Entire sample should be filtered (~3-4 mL).
      Tare microcentrifuge tube and weigh retentate. Add 1×PBS to retentate to add up to 1 mL.
   3. Centrifuge at 16,000 g for 1 hour @4 C.
   4. Freeze supernatant at −80 C.
An illustration is provided in FIG. 1 showing the flow of material from extraction through to isolation:

### Exosome Testing: CD63:

Exosome presence was confirmed via detection of transmembrane protein marker CD63, one of hallmarks of exosomes.
   Human CD63 ELISA Kit: Thermofisher Scientific Item #EH95RBX5
Table 1 contains a summary of the CD63 results
Conclusions:
   are detectable in both fresh cryopreserved bone tissue and devitalized lyopreserved bone tissue whereas little to no detectable exosomes were present in decellularized cancellous.

TABLE 1

| | | | Aveage |
|---|---|---|---|
| | | | Normalized |
| | | | Exosome |
| Tissue Form | Extraction Method | Purification | Content (0-1) |
| Positive Control | Collagenase | Centrifugal | 0.15 |
| Positive Control | Collagenase | 0.22 um filter | 0.15 |
| Positive Control | Collagenase | Centrifugal + | 0.09 |
| | | 0.22 um filter | |
| Devitalized Cancellous | Collagenase | Centrifugal | 0.32 |
| Devitalized Cancellous | Collagenase | 0.22 um filter | 0.97 |
| Devitalized Cancellous | Collagenase | Centrifugal + | 0.33 |
| | | 0.22 um filter | |
| Decellularized Cancellous | Collagenase | Centrifugal | 0.00 |
| Decellularized Cancellous | Collagenase | 0.22 um filter | 0.00 |
| Decellularized Cancellous | Collagenase | Centrifugal + | 0.02 |
| | | 0.22 um filter | |

| | | | Average |
|---|---|---|---|
| | | | Normalized |
| | | | Exosome |
| Tissue Form | Extraction Method | Purification | Content (0-1) |
| Positive Control | Collagenase + DNAse | Centrifugal | 0.17 |
| Positive Control | Collagenase + DNAse | 0.22 um filter | 0.13 |
| Positive Control | Collagenase + DNAse | Centrifugal + | 0.14 |
| | | 0.22 um filter | |
| Devitalized Cancellous | Collagenase + DNAse | Centrifugal | 0.03 |
| Devitalized Cancellous | Collagenase + DNAse | 0.22 um filter | 0.95 |
| Devitalized Cancellous | Collagenase + DNAse | Centrifugal + | 0.32 |
| | | 0.22 um filter | |
| Decellularized Cancellous | Collagenase + DNAse | Centrifugal | 0.00 |
| Decellularized Cancellous | Collagenase + DNAse | 0.22 um filter | 0.09 |
| Decellularized Cancellous | Collagenase + DNAse | Centrifugal + | 0.00 |
| | | 0.22 um filter | |

### Example 2

### Exosome Content Determined Based on CD63 for Cryopreserved, Devitalized, and Decellularized Human Cancellous Bone Compositions

### Donor Processing:

Fresh milled cancellous (post acetic acid and PBS rinses, milled to particle size range of about 212 μm to 2 millimeters (mm)) processed from donors within 72-hours post-mortem were received and split into two groups; as described in detail below, continuous processing was performed for fresh-tissue test groups and freeze/thaw processing was performed for devitalized and negative control/decellularized test groups.

### Tissue Groups & Descriptions:

Viable Cryopreserved Cancellous: fresh-donor derived cancellous tissue cryopreserved using controlled-rate freezing in cell culture media and 10% DMSO
1× Freeze/Thaw: fresh cancellous bone that has been frozen over 24 hrs and thawed for 4-5 hrs (1×) in 95% glycerol. This group was used to assess alternative interim preservation steps to maximize retention of exosomes and cells. This cancellous tissue was lyopreserved in 0.4M trehalose-saline solution
Devitalized Cancellous: fresh cancellous bone that has been frozen over 24 hrs and thawed for 4-5 hrs (2×) to mimic the stresses frozen donors would experience in processing. This mock frozen-donor derived cancellous tissue was lyopreserved in 0.4M trehalose-saline solution
Decellularized Cancellous: fresh cancellous bone that has been frozen over 24 hrs and thawed for 4-5 hrs (2×) to mimic the stresses frozen donors would experience in processing. The cancellous undergoes subsequent cleaning in mild surfactant, alcohol, and oxidizing agent. This mock frozen-donor derived and chemically processed cancellous tissue was lyopreserved in 0.4M trehalose-saline solution

### Exosome Extraction:

All test groups underwent the following extraction method:
   1. Collagenase & DNase—24 hr digestion in Collagenase II at 20 U/mL and DNAse at 40 U/mL in DMEM F12 and 15 mM HEPES

### Exosome Isolation:

After 24 hrs were complete, each test group underwent subsequent differential centrifugation steps and filtration via the proceeding methods:
Purification via Differential Centrifugation & Filtration:
   1. Centrifuge at 300 g for 10 minutes @4 C. Discard floating tissue and transfer supernatant to new 5 mL tubes.
   2. Centrifuge at 2000 g for 20 minutes @4 C. Transfer supernatant to new 5 mL tubes.
   3. Filter supernatant with 0.22 μm pore size syringe filters
   4. Centrifuge at 16,000 g for 1 hour @4 C.
   5. Freeze supernatant at −80 C.

### Exosome Testing: CD63:

Exosome presence was confirmed via presence of transmembrane protein marker CD63, one of hallmarks of exosomes.
   Human CD63 ELISA Kit: Thermofisher Scientific Item #EH95RBX5
Table 2 below contains a summary of the CD63 results.
Conclusions:
   Detectable levels of exosomes are retained in both viable cryopreserved and devitalized test groups that underwent one or two freeze-thaws.
   CD63 values are significantly higher in Devitalized Cancellous compared to Decellularized Cancellous bone.

TABLE 2

| | Average | |
|---|---|---|
| | Normalized | % Relative to |
| | Exosome | Devitalized |
| Test Groups | Content (0-1) | Cancellous |
| Viable Cryopreserved | 0.69 | +8% |
| Cancellous | | |
| 1X F/T | 0.44 | −29% |
| Devitalized Cancellous | 0.62 | N/A |
| Decellularized Cancellous | 0.00 | −100% |

### Example 3

### Bioavailable and Matrix-Bound Exosome Content Determined Based on CD81 for Human Devitalized Cancellous Bone Compositions, and Decellularized Human Cortical Bone Compositions

### Donor Processing:

1. Previously frozen donor bone was received, and excess soft tissue was debrided from the surface of the bone to leave behind cortical cross sections and osteochondral anatomical sites.
2. Osteochondral grafts as well as hemi-pelvis were dissected, removing the outer cortical wall and cutting the cancellous into blocks.
3. Cancellous blocks were soaked in basal media for 4 hours and subsequently milled to cancellous bone particles sized from about 212 μm to about 2 mm.
4. A portion of the milled cancellous bone was set aside and demineralized with hydrochloric acid over two soaks, the first for 2 hours followed by a 1.5 hour soak. The demineralized bone was subsequently rinsed with sodium dibasic buffer and water.
5. Long bone shafts were reamed to remove bone marrow, delipidized and milled into cortical bone fibers. A portion of the cortical bone fibers was kept aside to remain mineralized for testing.
6. Leftover cortical cross sections were milled into cortical powder. A portion of the powder was kept aside to remain mineralized for testing.
7. The mineralized cortical fibers and cortical powder were mixed and subsequently demineralized with hydrochloric acid for 15 mins. The demineralized cortical bone mixture was subsequently rinsed with sodium dibasic buffer and water.
8. All tissue samples were lyophilized with 0.4M trehalose in saline solution until dry.
9. Post-lyophilization, samples were extracted via a) enzymatic extraction with 20 U/mL collagenase II with DNAse and b) elution in DMEM over 24 hours
10. Exosome content in the bioavailable fraction (elution extraction of mineralized tissue) and matrix-bound fraction (enzymatic extraction of demineralized tissue) was quantified via CD81 ELISA Kit (Raybiotech, Ref ELH-CD81-1) and compared between the two test groups.

### Results

As shown in FIG. 2 and Table 3 below, exosomes are present and bioavailable in the mineralized cancellous component while also retaining a large portion of matrix-bound exomes available over the duration of healing. The levels of exosomes present are greater than non-cellular demineralized tissues such as demineralized cortical bone.

TABLE 3

| | Average CD81 Content (Normalized) | | |
|---|---|---|---|
| | Bioavailable | Matrix-bound | Sum |
| Devitalized Cancellous | 0.57 | 0.39 | 0.96 |
| Demineralized Cortical Tissue | 0.02 | 0.21 | 0.23 |

### Example 4

### Viability and Presence of Cellular Components Based on ATP for Fresh Control, Cryopreserved, and Devitalized Cancellous Bone Compositions

### Donor Processing:

Fresh milled cancellous bone particles (sized from 212 μm to 2 mm, and post acetic acid and PBS rinses) processed from donors within 72-hours post-mortem were received and split into two groups; continuous processing as described in detail below was performed for fresh-tissue test groups (pre-lyo/cryo cancellous and cryopreserved cancellous) and freeze/thaw was performed for devitalized cancellous test groups.

### Tissue Groups & Descriptions:

Pre-Lyo/Cryo Cancellous No soak: fresh donor-derived cancellous tissue post acetic acid and PBS rinses tested before preservation via lyophilization or cryopreservation. Used to establish a baseline or incoming (initial sample) viability reading.
Cryopreserved Fresh Cancellous: fresh-donor derived cancellous tissue cryopreserved in cell culture media with 10% DMSO.
Devitalized Cancellous 24 Hr Soak: fresh cancellous bone that has been frozen over 24 hrs and thawed for 4-5 hrs (2×). The cancellous tissue was then incubated in cell culture media for 24 hrs. After 24 hrs, cell culture media was decanted and this mock frozen-donor derived cancellous tissue was lyopreserved in 0.4M trehalose-saline solution.

### Sample Testing

ATP testing was performed using CellTiterGlo ATP Assay (test kit commercially available from Promega, located in Madison, WI, U.S.A). Briefly, all tested tissue samples were rinsed with saline and dried using gauze before being placed into pre-weighed weighing pans. Cryopreserved samples were thawed and decanted of cryopreservation solution and subsequently rinsed with saline whereas non-frozen or lyophilized samples were rinsed directly with saline before drying with gauze. The rinsed tissue samples were combined with an assay reagent and incubated on an orbital shaker. After incubation, aliquots of the incubated reagent were transferred into a 96-well plate and raw luminescence values (in RLU) were obtained using a plate reader. A standard curve was prepared by creating serial dilutions of known concentrations of an ATP standard, combining with assay reagent, incubating on an orbital shaker, and then reading the RLU of each dilution. The dilutions' RLUs were plotted against the known concentrations of each serial dilution to generate an equation, which was then used to convert RLU of the tissue samples to concentrations of ATP. Concentrations of ATP were corrected for by the weight in grams of each sample.
Retention of cells in the cancellous matrix was assessed using histological sectioning and staining for hematoxylin and eosin in order to visualize cell nuclei. A representative image of devitalized cancellous after 24 hr soak is provided in FIG. 4.

### Results

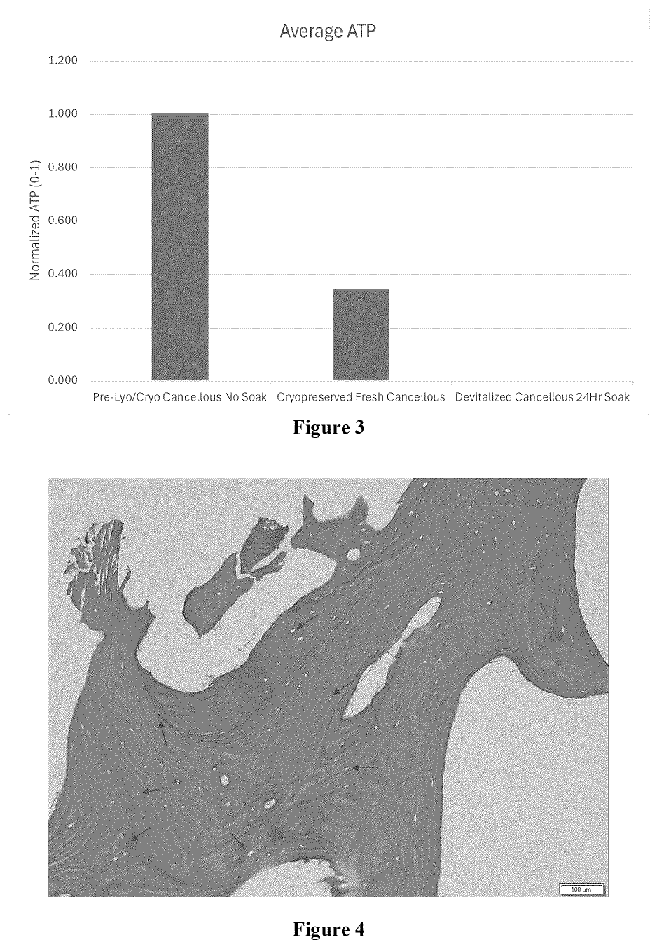

Compared to fresh cryopreserved and lyopreserved cancellous, devitalized tissue forms processed with two freeze thaw cycles and subsequent 24 hr soak were undetectable on viability assays via ATP, but cellular components (positively stained through hematoxylin) were still present/retained in the cancellous matrix. See FIG. 3.

TABLE 4

| | Average ATP |
|---|---|
| Test Group | (normalized) |
| Pre-Lyo/Cryo Cancellous No Soak | 1.000 |
| (incoming/initial baseline) | |
| Cryopreserved Fresh Cancellous | 0.345 |
| Devitalized Cancellous 24 Hr Soak | 0.000 |

### Example 5

Complete Devitalization Via Extended Soak Time Evaluated with Successful Devitalization of Cells and Retention of Devitalized Cells and Growth Factors Demonstrated.

### Donor Processing:

Fresh milled cancellous (post acetic acid and PBS rinses) and demineralized cortical fibers processed from donors within 72-hours post-mortem were split into two groups; continuous processing for fresh-tissue test groups and frozen for devitalized and decellularized test groups.
This example contains data from two unique donors.

### Tissue Groups & Descriptions:

Cryopreserved Fresh Bone Grafts: fresh-donor derived cancellous tissue incubated in cell culture media over 4 hrs, subsequently mixed with demineralized cortical fibers and cryopreserved in the cell culture media with 10% DMSO.
Devitalized Bone Grafts: fresh cancellous bone that has been frozen over 24 hrs and thawed for 4-5 hrs (2×) to mimic the stresses frozen donors would experience in processing. The cancellous tissue was then incubated in 1×PBS for 24 hrs. After 24 hrs, the cancellous and 1×PBS was mixed with demineralized cortical fibers that also underwent the same freeze/thaw cycles as the cancellous and subsequently lyopreserved in 0.4M trehalose-saline solution.
Decellularized: fresh cancellous bone that has been frozen over 24 hrs and thawed for 4-5 hrs (2×) to mimic the stresses frozen donors would experience in processing. The cancellous subsequently undergoes soaks in mild surfactant, ethanol, and an oxidizing agent to completely decellularize the tissue. After cleaning, cancellous was subsequently rinsed in several gallons of water, tap-dried, and added to 1×PBS. The cancellous and 1×PBS was mixed with demineralized cortical fibers that also underwent the same freeze/thaw cycles as the cancellous and subsequently lyopreserved in 0.4M trehalose-saline solution.

### Sample Testing

ATP testing was performed using CellTiterGlo ATP Assay (test kit commercially available from Promega, located in Madison, WI, U.S.A) as described in example #4.
Protein extractions were performed on each tissue group using elution and mechanical pulverization. Test samples were incubated in DMEM over 24 hrs at 4 C. After 24 hrs, samples were mechanically pulverized using RINO® bullet blender system (Next Advance) on the max setting and maximum time. Extracts were centrifuged to separate the tissue from solution, The retentate solution was pipetted into 5 mL tubes and frozen at −80 C until testing.
ELISAs were performed to assess levels of BMP-2, VEGF, and TGFb1 using R&D Systems Quantikine ELISA kits for each respective analyte of interest.

### Results

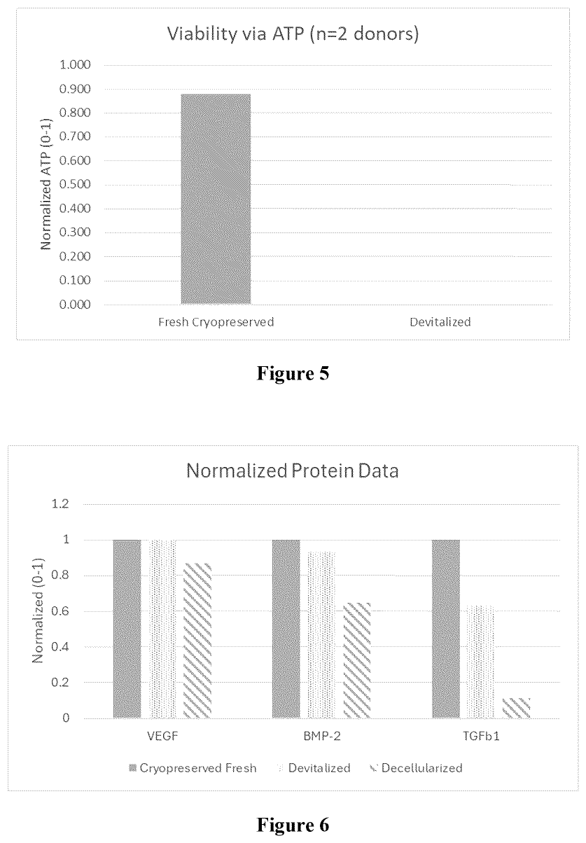

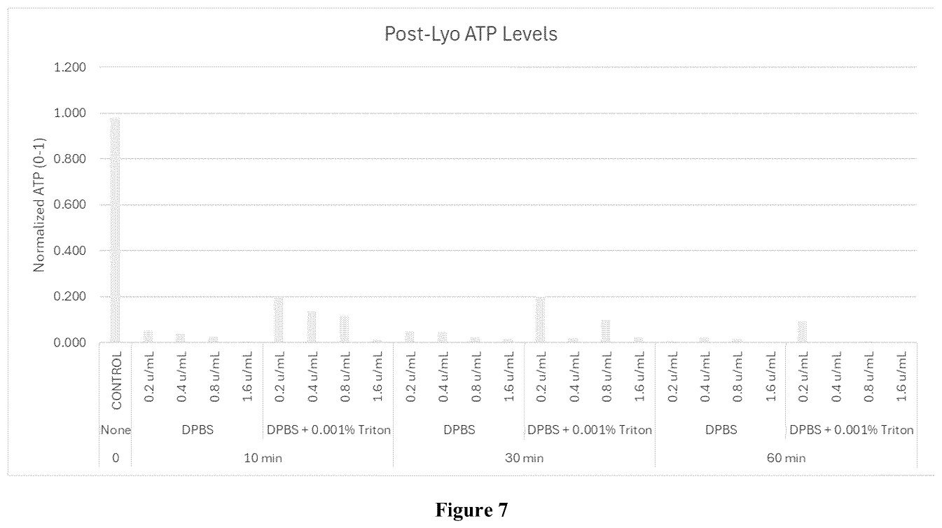

Results were averaged across two donors and normalized using min-max scaling.
The devitalized tissue forms processed with two freeze thaw cycles and subsequent 24 hr soak were determined to have 0 viability as measured by ATP with protein content at similar levels to fresh-cryopreserved counterpart and higher than decellularized graft. Results are presented in FIGS. 5 & 6

### Example 6

Devitalization of Cancellous Bone by Contact/Soaking with Apyrase in Various Concentrations

### Donor Processing:

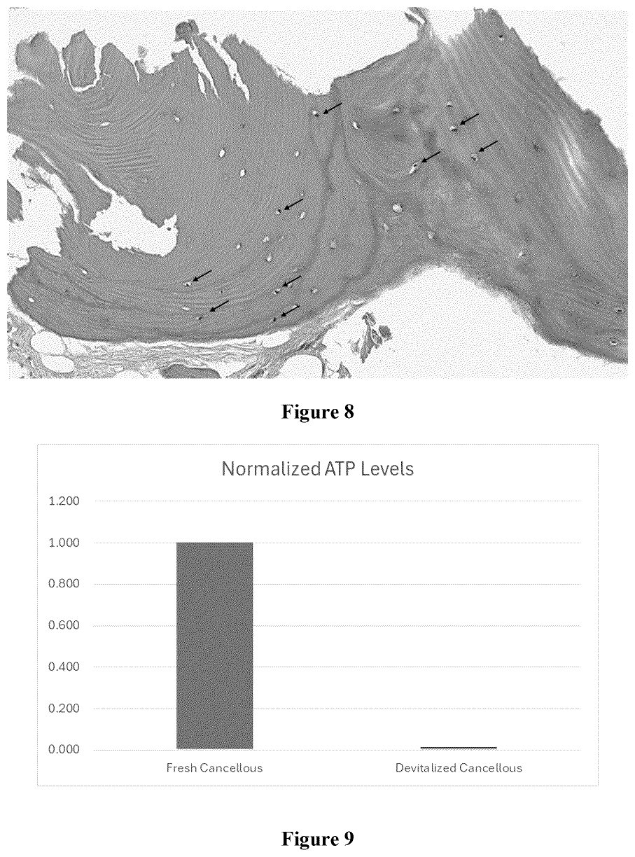

1. Fresh milled cancellous (post acetic acid and PBS rinses) processed from donors within 72-hours post-mortem was received and underwent subsequent processing steps, including contact with apyrase, to devitalize and retain cells.
2. Apyrase was received and diluted to final working concentrations of 0.2 U/mL, 0.4 U/mL, 0.8 U/mL, and 1.6 U/mL in either DPBS containing calcium and magnesium or DPBS containing calcium and magnesium and 0.001% Triton.
3. All apyrase-containing solutions were sterile-filtered.
4. Prior to any incubation or further processing, cancellous control samples were tested via CellTiter-Glo® ATP assay for incoming viability levels as a baseline. A matching set of control samples were set aside with 0.4M trehalose-saline solution and lyophilized to serve as post-lyo viable control samples.
5. All other samples were aliquot to 4-5 g of tissue per test group. Enough samples were made to accommodate all variations of apyrase in DPBS and apyrase in DPBS+0.001% Triton for incubation periods of 10, 30, and 60 mins.
6. After incubation, apyrase-containing solutions were decanted and pat-dry with gauze.
7. Cancellous samples were aliquotted into 1 oz jars with 4 mL of 0.4M trehalose-saline solution and lyophilized.
8. After the lyophilization cycle was completed, samples were tested for viability via CellTiter-Glo® ATP assay as described in the samples above for post-lyo viability with results summarized in Table 5 below and shown in FIG. 7.
9. Samples were sent out for histology to assess cell retention as described in the samples above, with several cells being identified as retained and undisrupted in the cancellous matrix as shown in FIG. 8.

### Results

Apyrase was able to successfully eliminate ATP which, in turn, devitalizes cells by inhibiting and/or preventing cellular metabolic activity (i.e., 0 ATP levels) from fresh-tissue donors while retaining the undisrupted devitalized native cells within the cancellous matrix post-processing and preservation.

TABLE 5
Post-Lyo ATP Testing

| | | | Normalized |
|---|---|---|---|
| Time | Incubation | Concentration | Average |
| (min) | Media | (u/mL) | ATP(0-1) |
| 0 | None | CONTROL | 0.983 |
| 10 min | DPBS | 0.2 u/mL | 0.056 |
| | | 0.4 u/mL | 0.040 |
| | | 0.8 u/mL | 0.028 |
| | | 1.6 u/mL | 0.009 |
| | DPBS + | 0.2 u/mL | 0.206 |
| | 0.001% | 0.4 u/mL | 0.137 |
| | Triton | 0.8 u/mL | 0.117 |
| | | 1.6 u/mL | 0.015 |
| 30 min | DPBS | 0.2 u/mL | 0.050 |
| | | 0.4 u/mL | 0.047 |
| | | 0.8 u/mL | 0.025 |
| | | 1.6 u/mL | 0.019 |
| | DPBS + | 0.2 u/mL | 0.201 |
| | 0.001% | 0.4 u/mL | 0.021 |
| | Triton | 0.8 u/mL | 0.101 |
| | | 1.6 u/mL | 0.023 |
| 60 min | DPBS | 0.2 u/mL | 0.008 |
| | | 0.4 u/mL | 0.023 |
| | | 0.8 u/mL | 0.016 |
| | | 1.6 u/mL | 0.000 |
| | DPBS + | 0.2 u/mL | 0.093 |
| | 0.001% | 0.4 u/mL | 0.005 |
| | Triton | 0.8 u/mL | 0.007 |
| | | 1.6 u/mL | 0.000 |

Note:
“Concentration (u/mL)” is Apyrase Concentration (units/mL)

### Example 7

Devitalization of Cancellous Bone by Freeze/Thaw and Apyrase Contact; Viability, Exosome and VEGF Content Measured for Fresh and Devitalized Cancellous Bone, with and without Demineralized Cortical Bone (Fibers)

### Donor Processing:

Fresh cancellous blocks processed from donors within 72-hours post-mortem were received and underwent subsequent processing steps (debriding, dissection, soaking in basal media, and milling, as described in Example 3 above) to create test groups that retain a population of viable cells post cryopreservation and another test group that contains non-viable cells after lyophilization i.e., devitalized.
Demineralized cortical fibers (DBFs) were processed from the same donor following conventional processing methods previously disclosed above (i.e., cortical bone was isolated and cleaned using detergents and/or surfactants to remove residual blood and lipids, then cut into cross-sectional segments of the appropriate length for milling, followed by milling of the shaft cross-sections to produce elongated fibers of cortical bone, which were demineralized in dilute acid to produce the demineralized cortical fibers). These DBFs were either tested alone or combined with cancellous to create cortico-cancellous test groups of viable fresh and devitalized samples.

### Tissue Groups & Descriptions:

Fresh Cancellous: Fresh-donor derived cancellous tissue cryopreserved in cell culture media and 10% DMSO
Fresh Cancellous & Fibers: Fresh-donor derived cancellous mixed in a 2:3 ratio with demineralized cortical fibers (g/g) cryopreserved in cell culture media and 10% DMSO.
Devitalized Cancellous: milled cancellous that has undergone two freeze-thaw cycles, a 30 minute apyrase soak, incubated in PBS for 4 hours, and lyophilized in 0.4M trehalose-saline solution.
   Apyrase was received and diluted to final working concentrations of 0.75 U/mL in DPBS containing calcium and magnesium. Apyrase solutions were then sterile filtered.
Devitalized Cancellous & Fibers: milled cancellous that has undergone two freeze-thaw cycles, a 30 minute apyrase soak, incubated in PBS for 1 hour, mixed in a 2:3 ratio with demineralized cortical fibers (g/g), and lyophilized in 0.4M trehalose-saline solution.

### Testing:

Cancellous-only groups were tested for viability via CellTiter-Glo® ATP assay.
All test groups were tested for presence of exosomes via CD63 ELISA and enzymatic extraction. Purification of exosomes was performed using ExoEasy Maxi Kit from Qiagen.
   Enzymatic extractions were performed using 20 U/mL collagenase II at 37 C over 24 hours.
   Elution was performed by incubating tissue in cell culture media over 24 hrs at 4 C.
Cancellous & fiber containing groups were tested for the presence of angiogenic protein VEGF via Quantikine ELISA kits from R&D Systems. Extractions were performed via elution or enzymatic methods as described above.

### Results

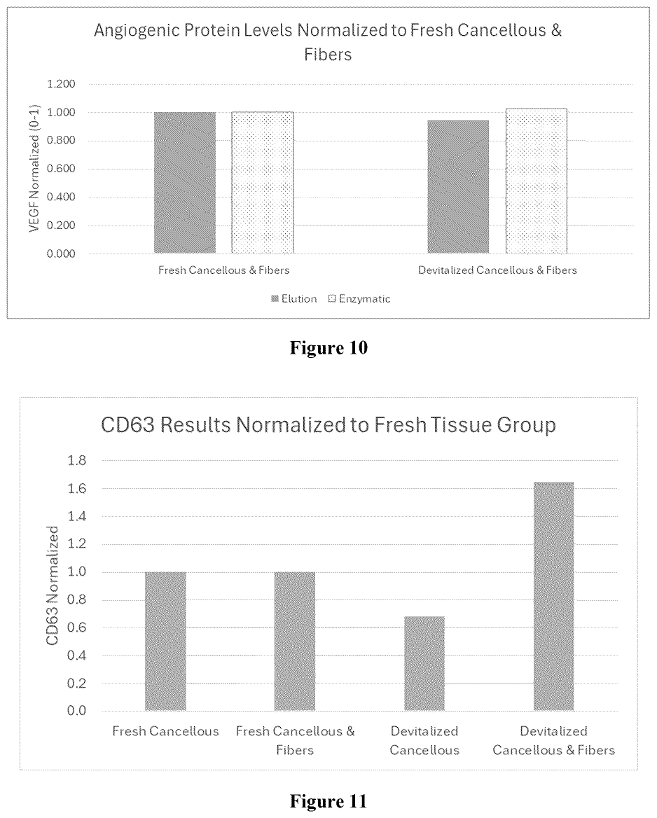

Cell viability results are summarized in Table 6 below and FIG. 9. As shown, viable cryopreserved cancellous with and without fibers have high detectable levels of ATP while devitalized tests groups after apyrase soak have little to no signal.
Angiogenic protein content did not differ between the two test groups as shown in Table 7 below and FIG. 10.
Exosome content via CD63 is highlighted in Table 8 below and shown in FIG. 11. Devitalized cancellous had similar levels of exosomes to the fresh viable tissue counterpart.
The results highlight that the same detectable levels of exosomes and protein content found in a viable cryopreserved graft can be retained in a devitalized non-viable tissue form following the processing described above.

TABLE 6

| ATP Normalized to | Average |
|---|---|
| Fresh Cancellous | ATP |
| Fresh Cancellous | 1.000 |
| Devitalized Cancellous | 0.014 |

TABLE 7
VEGF Content

| | | VEGF Content |
|---|---|---|
| Extraction | | Normalized to Fresh |
| Method | Sample | Cancellous & Fibers |
| Enzymatic | Fresh Cancellous & Fibers | 1.000 |
| | Devitalized Cancellous & Fibers | 1.026 |
| Mech Pulv | Fresh Cancellous & Fibers | 1.000 |
| | Devitalized Cancellous & Fibers | 0.943 |

TABLE 8

| | | CD63 Average |
|---|---|---|
| Extraction | | Normalized to Fresh |
| Method | Sample Description | Tissue Counterpart |
| Enz - Coll | Fresh Cancellous | 1 |
| | Fresh Cancellous & Fibers | 1 |
| | Devitalized Cancellous | 0.7 |
| | Devitalized Cancellous & Fibers | 1.6 |

### Example 8

Devitalization of Cancellous Bone by Freeze/Thaw and Apyrase Contact; Exosome and Growth Factor Content Measured, with and without Demineralized Cortical Bone (Fibers)

### Donor Processing:

Fresh cancellous blocks processed from donors within 72-hours post-mortem were received and underwent subsequent processing steps to create test groups that retain a population of viable cells post cryopreservation and another test group that contains non-viable cells after lyophilization i.e., devitalized.
Demineralized cortical fibers were processed from the same donor following conventional processing methods previously disclosed above (i.e., cortical bone was isolated and cleaned using detergents and/or surfactants to remove residual blood and lipids, then cut into cross-sectional segments of the appropriate length for milling, followed by milling of the shaft cross-sections to produce elongated fibers of cortical bone, which were demineralized in dilute acid to produce the demineralized cortical fibers (DBFs)). These fibers were either tested alone or combined with cancellous to create cortico-cancellous test groups of cryopreserved viable and devitalized samples.

### Procedure

1. Cancellous blocks recovered from donors within 72-hours post-mortem and subsequently milled, rinsed under agitation with PBS and acetic acid, and combined with demineralized cortical fibers from the same donor. The mix of cortico-cancellous tissue was subsequently cryopreserved in 10% DMSO representing the “cryopreserved viable” test group.
   A portion of cancellous tissue samples were demineralized in 0.6N HCl, mixed with fibers, and cryopreserved in 10% DMSO representing a demineralized formulation.
2. Remaining cancellous blocks and fibers were subsequently frozen at −80° C.
3. The remaining cancellous blocks and demineralized cortical fibers were then removed from −80° C. and allowed to thaw over a 4-5 hour period, representing the first freeze thaw in the process.
4. The cancellous blocks and demineralized cortical fibers were returned to a −80° C. freezer overnight (>24 hrs) and once more thawed over a 4-5 hour period, representing the second freeze thaw in the process, mimicking the stresses donors undergo as part of standard processing.
5. Post second freeze-thaw, cancellous blocks were milled and subsequently rinsed under agitation PBS, acetic acid, and apyrase.
6. Milled cancellous was then placed in a static PBS soak for 60 minutes.
7. Post-static-soak, cancellous tissue along with PBS was mixed with demineralized cortical fibers, aliquot into 1 oz jars with 0.4M trehalose-saline solution to cover the tissue, and lyophilized.
   A portion of cancellous tissue samples were demineralized in 0.6N HCl, mixed with fibers, and lyopreserved in 0.4M trehalose-saline representing a demineralized formulation.
8. A set of cortical-fiber only samples were aliquot into 1 oz jars with 0.4M trehalose-saline solution to cover the tissue, and lyophilized.

### Tissue Groups & Descriptions:

Cryopreserved Viable: Fresh-donor derived cancellous mixed in a 1:1 ratio with demineralized cortical fibers (g/g) cryopreserved in cell culture media and 10% DMSO.
Devitalized Cancellous & Fibers: milled cancellous that has undergone two freeze-thaw cycles, a 30-minute apyrase soak, incubated in PBS for 1 hour, mixed in a 1:1 ratio with demineralized cortical fibers (g/g), and lyophilized in 0.4M trehalose-saline solution.
   Apyrase was received and diluted to final working concentrations of 0.75 Units/mL in DPBS with calcium and magnesium. Apyrase solutions were then sterile filtered.
Fibers: demineralized cortical fibers only

### Sample Testing

To assess the osteoinductive, angiogenic, and anti-inflammatory potential of the different tissue groups, ELISA kits for BMP-2, VEGF, and TGFb1 respectively from R&D Systems (Minneapolis, MN) were used for testing extracts.
   To extract bioavailable proteins, elution was performed in Dulbecco's Modified Eagle Medium (DMEM) over 24 hrs at 4° C.
   To extract matrix-bound proteins, the demineralized formulation of each test group was extracted via enzymatic digestion using Gibco's (Waltham, MA) collagenase II over 24 hrs at 37° C.
   Each test group had a total of three samples tested across three different donors. Normalized results across three donors are presented below, ranging from 0-1 following standard min-max normalization across all test sample captured in Tables 9-11.
To assess the retention of cell-derived exosomes and cell-associated elements, a multiplex array from Eve Technologies (Calgary, Canada) was utilized.
   To extract cell-derived exosomes and cell-associated elements, elution was performed in Dulbecco's Modified Eagle Medium (DMEM) over 24 hrs at 4° C.
   Exosome purification was performed first by differential centrifugation followed by isolation using an exosome purification kit from Qiagen (Hilden, Germany).
   Each test group had a total of three samples tested across three different donors. Results across three donors are presented below, ranging from 0-1 following standard min-max normalization across the elution captured in Tables 12-14.

### Results

The devitalized tissue form had levels of osteoinductive, angiogenic, and anti-inflammatory proteins at similar levels to fresh cryopreserved viable tissue and higher than that of standard demineralized bone fibers.
   For BMP-2, only matrix-bound extractions were tested and analyzed
The devitalized tissue form had similar levels of cell-derived exosomes/exosomes markers (CD63, TSG101) and cell-associated elements (calreticulin) to fresh cryopreserved viable tissue and significantly higher than that of standard demineralized bone fibers.

TABLE 9

Matrix-Bound

| Average | | Devitalized | |
|---|---|---|---|
| Normalized | Cryopreserved | Cancellous | |
| BMP-2 | Viable | & Fibers | Fibers |
| Donor 1 | 0.79 | 0.88 | 0.93 |
| Donor 2 | 0.73 | 0.45 | 0.88 |
| Donor 3 | 0.72 | 0.93 | 0.71 |
| Average | 0.75 | 0.75 | 0.84 |
| Stdev | 0.04 | 0.26 | 0.11 |

TABLE 10

| | | | | Devitalized | | | | | |
|---|---|---|---|---|---|---|---|---|---|
| Average | Cryopreserved Viable | | | Cancellous & Fibers | | | Fibers | | |
| Normalized | Bio- | Matrix- | | Bio- | Matrix- | | Bio- | Matrix- | |
| VEGF | available | Bound | Composite | available | Bound | Composite | available | Bound | Composite |
| Donor 1 | 0.31 | 0.82 | 1.13 | 0.20 | 0.50 | 0.70 | 0.00 | 0.79 | 0.79 |
| Donor 2 | 0.53 | 0.94 | 1.47 | 0.47 | 0.82 | 1.29 | 0.00 | 0.90 | 0.90 |
| Donor 3 | 0.41 | 0.81 | 1.21 | 0.56 | 0.87 | 1.43 | 0.00 | 0.94 | 0.94 |
| Average | 0.42 | 0.86 | 1.27 | 0.41 | 0.73 | 1.14 | 0.00 | 0.88 | 0.88 |
| Stdev | 0.11 | 0.07 | 0.19 | 0.19 | 0.19 | 0.37 | 0.00 | 0.11 | 0.11 |

TABLE 11

| | | | | Devitalized | | | | | |
|---|---|---|---|---|---|---|---|---|---|
| Average | Cryopreserved Viable | | | Cancellous & Fibers | | | Fibers | | |
| Normalized | Bio- | Matrix- | | Bio- | Matrix- | | Bio- | Matrix- | |
| TGFb1 | available | Bound | Composite | available | Bound | Composite | available | Bound | Composite |
| Donor 1 | 0.0 [symbol] | 1.00 | 1.0 [symbol] | 0.03 | 0.91 | 0.94 | 0.00 | 0.61 | 0.61 |
| Donor 2 | 0.0 [symbol] | 1.00 | 1.0 [symbol] | 0.11 | 0.81 | 0.91 | 0.00 | 1.00 | 1.00 |
| Donor 3 | 0.04 | 1.00 | 1.04 | 0.11 | 0.89 | 1.00 | 0.00 | 0.58 | 0.58 |
| Average | 0.0 [symbol] | 1.00 | 1.05 | 0.08 | 0.87 | 0.95 | 0.00 | 0.73 | 0.73 |
| Stdev | 0.01 | 0.00 | 0.01 | 0.04 | 0.05 | 0.04 | 0.00 | 0.24 | 0.24 |

[symbol]

indicates data missing or illegible when filed

TABLE 12

Bio-available

| Average | | Devitalized | |
|---|---|---|---|
| Normalized | Cryopreserved | Cancellous | |
| Calreticulin | Viable | & Fibers | Fibers |
| Donor 1 | 1.00 | 0.71 | 0.00 |
| Donor 2 | 0.72 | 1.00 | 0.00 |
| Donor 3 | 1.00 | 0.68 | 0.00 |
| Average | 0.91 | 0.80 | 0.00 |
| Stdev | 0.16 | 0.18 | 0.00 |

TABLE 13

Bio-available

| Average | | Devitalized | |
|---|---|---|---|
| Normalized | Cryopreserved | Cancellous | |
| TSG101 | Viable | & Fibers | Fibers |
| Donor 1 | 0.00 | 1.00 | 0.00 |
| Donor 2 | 1.00 | 0.32 | 0.00 |
| Donor 3 | 0.97 | 1.00 | 0.00 |
| Average | 0.66 | 0.77 | 0.00 |
| Stdev | 0.57 | 0.40 | 0.00 |

TABLE 14

Bio-available

| Average | | Devitalized | |
|---|---|---|---|
| Normalized | Cryopreserved | Cancellous | |
| CD63 | Viable | & Fibers | Fibers |
| Donor 1 | 1.00 | 0.73 | 0.00 |
| Donor 2 | 0.94 | 1.00 | 0.00 |
| Donor 3 | 0.74 | 1.00 | 0.00 |
| Average | 0.89 | 0.91 | 0.00 |
| Stdev | 0.14 | 0.15 | 0.00 |

### Example 9

### Clinical Study (Rat)—Assess Capability of Devitalized Composition (Devitalized Cancellous Bone & Demineralized Cortical Bone Fibers) to Promote New Bone Formation

### Donor Processing:

Devitalized test groups samples were processed according to the exemplary methods described above. Whole donor tissue was recovered from donors and underwent two freeze-thaw cycles. Cortical tissue was processed into demineralized cortical fibers. Cancellous tissue was cut, milled, rinsed under agitation with PBS, acetic acid, and apyrase and underwent a static soak in PBS for one hour. PBS, cortical tissue, and cancellous tissue were mixed until homogenous, aliquot into 1 oz or 2 oz jars with 0.4M trehalose-saline solution to cover the tissue, and subsequently lyophilized.

### Tissue Groups & Descriptions:

Devitalized Cancellous & Fibers: milled cancellous that has undergone two freeze-thaw cycles, a 30-minute apyrase soak, incubated in PBS for 1 hour, mixed in a 1:1 ratio with demineralized cortical fibers (g/g), and lyophilized in 0.4M trehalose-saline solution.
   Apyrase was received and diluted to final working concentrations of 0.75 U/mL in DPBS with calcium and magnesium. Apyrase solutions were then sterile filtered.

### Sample Testing—Athymic Rat Posterolateral Fusion (PLF) Model:

To evaluate the capability of a devitalized graft to create bone, a single-level posterolateral spine fusion in an athymic nude rat model was employed. Devitalized cancellous & fiber samples and syngeneic bone control groups were implanted across the transverse processes and lateral gutters in the 4^{th} and 5^{th} lumbar vertebrae (L4-L5) and outcome measures of manual palpation were tested at 5 weeks. All devitalized test group articles were processed aseptically with donor lot passing USP<71> sterility testing prior to implantation.
Approximately 0.3 cc of test materials were deployed onto the paraspinal beds bridging the decorticated transverse processes, followed by wound closure with standard surgical techniques. Radiography was employed immediately after surgery to verify implant placement. Animals were recovered from surgery with appropriate analgesic and post-operative care. All animals completed the study with normal weight gain with no overt signs of toxicity and were clinically healthy for the study duration.
At 5 weeks±1 day post-operatively the rats were weighed and then euthanized in a carbon dioxide (CO2) induction chamber. The lumbar spines were explanted and the operated segments were then palpated for motion by at least two independent evaluators who remained blind to group designations. Palpation preceded by grasping the spine at L5 with the thumb and index finger of the right hand and L4 with the thumb and index finger of the left hand. Lateral pressure was then applied in one direction and then the other and the presence of motion in the segment was determined by direct visualization. Dorsal/ventral pressure was then similarly applied. Similar procedures were applied to adjacent non-operated spaces for comparison purposes. Operative segments that demonstrate no reduction in the range of motion compared to adjacent non-operated segments were scored as “0”. Segments that demonstrate a >50% reduction of range of motion compared to adjacent segments were scored as “1”. Segments that demonstrated a solid fusion were scored as “2”. Each side was evaluated, and scores were recorded for the right and the left side at each level. A summary of results is found in Table 15.

### Results

At the 5-week timepoint, the devitalized cancellous & fiber test group achieved a fusion rate of 83.3% by manual palpation. Five out of six rats scored a “2” indicating solid fusion for both segments. Despite not scoring as a solid fusion, the remaining rat scored a “1” for both segments.

TABLE 15
Devitalized Cancellous & Fibers

Palpation L4-5

| Animal # | Donor # | Left | Right |
|---|---|---|---|
| 1 | Donor 1 | 2 | 2 |
| 2 | Donor 1 | 1 | 1 |
| 3 | Donor 2 | 2 | 2 |
| 4 | Donor 2 | 2 | 2 |
| 5 | Donor 3 | 2 | 2 |
| 6 | Donor 3 | 2 | 2 |

We claim:

1. A devitalized tissue-derived composition derived from one or more tissue samples which include one or more tissue types, the devitalized tissue-derived composition comprising:
extracellular matrix (ECM) endogenous to and retained from the one or more tissue samples, and either:
one or more endogenous bioactive substances which are retained from the one or more tissue samples and resident in the ECM; or
a population of devitalized endogenous cells, which are retained from the one or more tissue samples and are undisrupted and resident in the ECM; or
both of said one or more endogenous bioactive substances and said devitalized endogenous cells.

2. The devitalized tissue-derived composition of claim 1, wherein the endogenous bioactive substances comprise cellular components, bioactive factors, or combinations thereof.

3. The devitalized tissue-derived composition of claim 2, wherein the cellular components comprise one or more of: cytoplasm, cell membranes, nuclei, deoxyribonucleic acid (DNA), ribosomes, ribonucleic acid (RNA), and vesicles.

4. The devitalized tissue-derived composition of claim 2, wherein the bioactive factors include one or more of: growth factors and cytokines.

5. The devitalized tissue-derived composition of claim 1, wherein the endogenous cellular components are present in the tissue-derived composition in a proportion of at least 10%, by weight (wt %), based on the total weight of endogenous cellular components present in unprocessed tissue samples which were processed to produce the tissue-derived compositions.

6. The devitalized tissue-derived composition of claim 1, wherein at least a portion of the devitalized tissue-derived composition is lyophilized.

7. The devitalized tissue-derived composition of claim 1, further comprising one or more additional components which are biocompatible and selected from: processed or unprocessed natural materials, synthetic materials, textiles, carriers, and combinations thereof.

8. The devitalized tissue-derived composition of claim 7, wherein the one or more tissue samples include a first tissue type, and wherein the one or more additional components comprise processed or unprocessed natural materials comprising one or more tissue-derived matrices derived from one or more different tissue samples which include at least a second tissue type which is different from the first tissue type.

9. The devitalized tissue-derived composition of claim 1, wherein the one or more tissue samples included one or more cancellous bone samples and the tissue-derived composition contains endogenous cellular components retained from the one or more cancellous bone samples.

10. The devitalized tissue-derived composition of claim 9, wherein the one or more endogenous bioactive substances comprise extracellular vesicles and one or more growth factors retained from the one or more cancellous bone samples and wherein the devitalized tissue-derived composition promotes or enhances, after administration to a recipient, bone healing potential, angiogenic potential, osteoimmunomodulatory (OIM) potential, or a combination thereof.

11. A method for producing a devitalized tissue-derived composition which comprises one or more endogenous bioactive substances which were retained from one or more tissue samples processed to produce the devitalized tissue-derived composition and, optionally, a population of devitalized endogenous cells which were retained from the one or more tissue samples and which are undisrupted and resident in the ECM, the method comprising the steps of:
devitalizing endogenous cells of the one or more tissue samples, or a tissue-derived composition produced therefrom, by performing one or more technique one or more times and selected from:
performing at least one freeze-thaw cycle on the one or more tissue samples, degrading or destroying at least a portion of adenosine triphosphate (ATP) present in the one or more tissue samples, and
sterilizing the one or more tissue samples by exposing to radiation or ethylene oxide, wherein a devitalized tissue-derived composition is produced.

12. The method of claim 11, wherein each freeze/thaw cycle performed comprises freezing the one or more tissue bone sample and then at least partially thawing the frozen one or more tissue samples, wherein the freeze-thaw cycles are preferably performed under conditions which minimize or avoid lysis or other damage to cellular membranes.

13. The method of claim 11, wherein the step of degrading or destroying ATP comprises exposing, contacting, incubating, soaking, or a combination thereof, the one or more tissue samples to reagents capable of degrading or hydrolyzing ATP.

14. The method of claim 13, wherein the reagents comprise one or more enzymes.

15. The method of claim 11, wherein the step of devitalizing endogenous cells comprises performing at least one freeze-thaw cycle on the one or more tissue samples, and degrading or destroying at least a portion of the ATP by exposing, contacting, incubating, soaking, or a combination thereof, the one or more tissue samples to reagents comprising one or more enzymes.

16. The method of claim 11, further comprising combining one or more additional components to the devitalized tissue-derived composition, wherein the one or more additional components are biocompatible and selected from: processed or unprocessed natural materials, synthetic materials, textiles, carriers, and combinations thereof.

17. The devitalized tissue-derived composition of claim 16, wherein the one or more tissue samples include a first tissue type, and wherein the one or more additional components comprise processed or unprocessed natural materials comprising one or more tissue-derived matrices derived from one or more different tissue samples which include at least a second tissue type which is different from the first tissue type.

18. A method for producing devitalized tissue-derived compositions from one or more tissue samples comprising a cancellous bone sample comprising the steps of:
devitalizing native cells of the cancellous bone sample or a tissue-derived composition produced therefrom by performing one or more techniques one or more times and selected from:
a. prior to any processing steps, either:
(i) subjecting the cancellous bone sample to at least one freeze/thaw cycle, each of which comprises freezing the cancellous bone sample; or
(ii) subjecting the cancellous bone sample to irradiation; or
(iii) performing a combination of both (i) and (ii); and
b. after at least one size reduction step has been performed on the cancellous bone sample, either:
(i) subjecting the cancellous bone sample to a reagent or an enzyme capable of degrading, destruction, or both, of adenosine triphosphate (ATP) present in the cancellous bone sample, or;
(ii) incubating processed cancellous bone sample in a biologically compatible fluid to hydrolyze ATP and its derivatives; or
(iii) performing a combination of (i) and (ii) and
c. optionally, after all processing steps have been performed, subjecting a cancellous bone-derived composition produced from the cancellous bone sample to terminal sterilization by exposing the cancellous bone-derived composition to radiation or ethylene oxide.

19. The method of claim 18, further comprising obtaining demineralized cortical bone fibers and combining the demineralized cortical bone fibers with the devitalized cancellous bone granules.

20. The method of claim 18, wherein the step of (i) subjecting the cancellous bone sample to at least one freeze/thaw cycle comprises performing at least two freeze/thaw cycles, and the one or more enzymes capable of degrading, destruction, or both, of adenosine triphosphate (ATP) include apyrase.
